(19) United States
(12) Patent Application Publication
(10) Pub. No.: US 2018/0279992 A1
(43) Pub. Date: Oct. 4, 2018

(54) POSITIONING DEVICE AND METHOD OF USE

(71) Applicants: Cedars-Sinai Medical Center, Los Angeles, CA (US); Baker Group, LLP, Portland, OR (US)

(72) Inventors: Jonathan Frankel, Los Angeles, CA (US); Michael Baker, Portland, OR (US)

(73) Assignees: Cedars-Sinai Medical Center, Los Angeles, CA (US); Baker Group, LLP, Portland, OR (US)

(21) Appl. No.: 15/763,628

(22) PCT Filed: Sep. 30, 2016
(86) PCT No.: PCT/US2016/054970

§ 371 Date: Apr. 6, 2017

Related U.S. Application Data
(60) Provisional application No. 62/235,347, filed on Sep. 30, 2015.

(51) Int. Cl. A61B 8/00; A61G 13/10; A61M 37/00; A61B 90/50; B25J 19/00; B25J 9/16

(52) U.S. Cl. CPC A61B 8/4218; A61G 13/101; A61M 37/0092; A61B 90/50; F16M 13/027; B25J 9/1689; A61B 2090/5025; A61B 2090/508; A61B 2090/506; B25J 19/0004

(57) ABSTRACT

Aspects of the present disclosure include embodiments of a positioning device and a method for using the same. The method may comprise applying pressure during an ultrasound examination. Embodiments of the disclosed positioning device include an arch configuration and an arm configuration to span across a gurney and over a patient on the gurney, the arch including a track that spans at least part of a length the arch, and an instrument attachment attached to the track of the arch, or an arm with multiple joints with an instrument attachment attached to the distal end of the arm, which serves to assist the motions of the user. The instrument attachment is configured to hold an instrument, such as an ultrasound transducer, and may extend the instrument toward the patient, such as to apply pressure to the patient during an ultrasound examination.

### CROSS REFERENCE TO RELATED APPLICATION

This claims the benefit of U.S. provisional patent application No. 62/235,347, filed Sep. 30, 2015, which is incorporated herein by reference in its entirety.

### FIELD

The present invention relates to devices used in medical examinations and/or procedures. More particularly, the present invention concerns embodiments of a device, and a method for using the device, to position a medical instrument, such as an ultrasound transducer, relative to a patient during medical examinations and/or procedures.

### BACKGROUND

Ultrasound imaging is a critical component of the diagnostic and therapeutic tools available to modern medicine. However, the repetitive nature and inherent mechanics of ultrasonography cause stress injuries that limit the short and long term productivity of sonographers. Further, the examination quality suffers because of the fatigue and discomfort experienced by a sonographer while performing an examination.

The actions implicated as the primary mechanisms of injury are shoulder flexion and adduction, sustained static force, and awkward angled pinch grips on the ultrasound transducer. These mechanisms may cause a variety of injuries, including, but not limited to, tenosynovitis, shoulder bursitis, tennis or golfer's elbow, thoracic outlet syndrome, rotator cuff tears, and degenerative disc disease. The Center for Disease Control and Prevention (CDC), Occupational Safety and Health Administration (OSHA), and National Institute for Occupational Safety and Health (NIOSH) have made national recommendations and initiatives educating the community on the problem and palliative measures to reduce or prevent these injuries. According to OSHA, hospitals are required to pursue the greatest possible means to satisfy the injurious practices in their establishment or face fines. The only options thus far have been the palliative efforts provided by OSHA and the CDC. These efforts are not consistently applied by sonographers and their institutions due to their consequence on examination efficiency and ease.

Researchers argue that the cost to a hospital from a single sonographer who must take a leave from work due to stress-related injury could be as costly as $190,000. This estimate does not include indirect costs that can more than triple that amount to almost $600,000 when considering recruiting new employees, training costs, overtime, and temporary coverage expenses. The most concerning factor for hospitals is the frequency of this issue. Studies have shown that more than 80% of sonographers are scanning in pain and 20% will suffer a career ending injury. These injuries are not only common but develop rapidly causing pain within five years of a sonographer starting professional work.

Currently, sonographers have limited tools at their disposal to minimize the risk of repetitive stress injuries while performing their duties. They are educated and advised to use ergonomic chairs, vary their case load during the day, ensure proper posture and alignment with the patients during procedures, and pace their work to accommodate adequate break time for stretching and recuperation. All of these interventions are merely palliative measures that have failed to address the etiology of the primary injury. Worse yet, sonographers often find themselves in settings that are suboptimal for even applying these palliative measures because employing such measures reduces their performance efficiency.

### SUMMARY

According to the foregoing issues, a need exists for reducing or eliminating the mechanisms of injury afflicting medical workers, such as sonographers, thereby obviating the need for the above-mentioned palliative efforts. Disclosed herein are embodiments of a positioning device that can help reduce and/or eliminate injuries to medical professionals, such as sonographers. In some embodiments, the device comprises a plurality of locking mechanisms that are configured to inhibit and/or substantially prevent movement of an instrument assembly relative to a patient. As used herein the term “locking mechanism” refers to a mechanism that can dampen movement, lock the mechanism to prevent movement, and/or unlock the mechanism to allow movement. The instrument assembly may comprise an instrument attachment, configured to receive a desired instrument for a particular examination and/or procedure. The instrument attachment may be a transducer attachment, a needle attachment, a catheter attachment, a syringe attachment, a blunt instrument attachment, or a clamp and/or strap attachment, such as for use for traction and/or immobilization. In certain embodiments, the instrument attachment is a transducer attachment configured to receive a transducer.

The device also comprises at least one controller connected to the plurality of locking mechanisms, the controller configured to lock and unlock the locking mechanisms. The device may further comprise a support apparatus. In some embodiments, the support apparatus is or comprises an arm comprising at least one arm segment. In other embodiments, the support apparatus is or comprises an arch that may span the width of a gurney or bed. In some embodiments where the support apparatus is an arm, the instrument assembly is attached to a distal end of the support apparatus. The locking mechanisms may comprise a pneumatic locking mechanism, a wrap-spring locking mechanism, an electric actuator, or a combination thereof, and in certain embodiments, the locking mechanisms are pneumatic locking mechanisms. The locking mechanisms may have a default, rest or inactivated state that is an unlocked state that permits movement of the instrument assembly relative to a subject or patient. Activating the controller(s) activates the locking mechanisms to inhibit and/or substantially prevent movement of the instrument assembly relative to a subject or patient. Alternatively, the locking mechanisms may have a default, rest or inactivated state that is a locked state that inhibits and/or prevents movement of the instrument assembly relative to a subject or patient. In such embodiments, activating the controller(s) unlocks the locking mechanisms, thereby allowing the instrument assembly to move relative to the subject or patient.

The support apparatus may comprise a ceiling mount, a floor mount, a stand, a wall mount, a desk mount, a cabinet mount, or a bed or gurney mount, and in certain embodiments, the support apparatus comprises a ceiling mount. The support apparatus may comprise a mount that can attach to a medical machine, such as an ultrasound machine. The support apparatus may also comprise a first plurality of arm segments, which may be arranged sequentially. Alternatively, the support segment may comprise a plurality of ball and socket joints. The instrument assembly may be attached to a distal end of the first plurality of arm segments.

The instrument assembly may comprise a second plurality of arm segments, which may be arranged sequentially. The instrument attachment, such as a transducer attachment, may be connected to a distal end of the second plurality of arm segments.

The plurality of locking mechanisms are connected to at least one controller. In some embodiments, a single controller is connected to the plurality of locking mechanisms, but in other embodiments, two or more controllers are used. The plurality of locking mechanisms may comprise a first group of locking mechanisms and a second group of locking mechanisms. In some embodiments, the first group of locking mechanisms is connected to a first controller, and a second group of locking mechanisms is connected to a second controller. The first group of locking mechanisms may be connected to the support apparatus, and/or the second group of locking mechanisms may be connected to the instrument assembly, such as to the second plurality of arm segments. The at least one controller may comprise a pedal that is connected to at least a first portion of the locking mechanisms; a switch that is connected to at least a second portion of the plurality of locking mechanisms, such as on the instrument assembly; a computer controller that is connected to at least a third portion of the plurality of locking mechanisms, or a combination thereof.

In other embodiments, the support apparatus is an arch configured to span a width of a gurney, and over a patient. The arch may comprise a track that spans at least a part of the arch, and may reach from one side of the gurney to the other side. The instrument assembly may be connected to the track.

The arch may be configured to move relative to the gurney. The arch also may comprise connection points that attach the arch to gurney attachment rods located on either side of the gurney. At least one of the connection points may be configured to pivot the arch vertically, horizontally, or a combination thereof, to provide access for the patient to the gurney. Additionally, or alternatively, the arch may be configured to tilt superiorly towards a superior position of the gurney, inferiorly towards an inferior position of the gurney, or a combination thereof.

In any embodiments, the instrument assembly may be configured to extend the instrument attachment, such as the transducer attachment, and thereby an instrument, such as a transducer, attached thereto, to contact a patient. The quantity of pressure applied to the patient by the extension of the instrument attachment by the instrument assembly may be controlled and dictated by the amount of pressure applied by the user to a controller. The instrument assembly may comprise a first electrical actuator, a first pneumatic actuator, a first hydraulic actuator, a first electrical motor, or a combination thereof, configured to extend the instrument attachment toward, and to contact, the patient. The instrument assembly may also comprise a second electrical actuator, a second pneumatic actuator, a second hydraulic actuator, a second electrical motor, or a combination thereof, configured to rotate, tilt, or a combination thereof, the instrument attachment relative to the patient, to position an instrument in a desired position and orientation relative to the patient.

In some embodiments, where the support apparatus is an arch, the at least one controller comprises a pedal in communication with the locking mechanisms, and configured to dampen, lock and/or unlock the locking mechanisms. The locking mechanisms may be located at the intersection points, i.e. joints, where the degrees of freedom are located. The pedal may be connected to an actuator and/or electric motor on the instrument assembly, to extend the instrument attachment toward the patient. The pedal may be configured to sequentially dampen and lock the arch and the instrument assembly, as the pedal is depressed. Alternatively, the arch and the instrument assembly have a default locked position and the pedal is configured to sequentially unlock the arch and the instrument assembly as the pedal is released.

In any embodiments, the instrument attachment, such as a transducer attachment, may comprise pliable bands, ratcheting straps, a threaded clamp, a hook and eye attachment, a snap molding, a friction material, or a combination thereof. The snap molding may be a preformed plastic packaging snap molding that the instrument, such as a transducer, is pressed into and grips the instrument through pressure from the surrounding mold. This way instruments are easily swapped in and out in a single motion.

Also disclosed is a method of using the device. In some embodiments, the method comprises positioning an instrument, such as a transducer, located in the instrument attachment in a desired position and orientation relative to a patient, and locking the plurality of locking mechanisms to substantially prevent movement of the instrument relative to the patient. Positioning the instrument may comprise applying pressure to the patient with the instrument, and locking the plurality of locking mechanisms substantially prevents a reduction of the pressure. Applying pressure may comprise manually applying pressure with the instrument. Alternatively, applying pressure may comprise activating the instrument assembly to move the instrument attachment to contact the patient with a pressure suitable for the application. In certain embodiments, the instrument is a transducer. In such embodiments, applying pressure may comprise manually applying pressure with the transducer, or activating the instrument assembly to move the transducer to contact the patient with a pressure suitable for obtaining an image.

An additional aspect of the present disclosure includes a method of applying pressure during an ultrasound examination. The method includes manipulating a positioning device with respect to a plurality of degrees of freedom relative to a patient. Degrees of freedom being the independent parameters that define an object's position and orientation, a rigid object in space typically has 6 degrees of freedom, one in each of the x, y, and z axes, and rotation about each of those axes. Movement of the object is therefore movement in one, or a combination of more than one, of these degrees of freedom. As used herein a degree of motion is the same as a degree of freedom. The method further includes dampening and/or locking the motion of the positioning device relative to one or more of the plurality of degrees of freedom during pressure. Optionally, the method may further include extending an instrument attachment towards the patient to apply pressure to the patient with a transducer during the ultrasound examination through the use of a controller, such as a pedal.

The foregoing and other objects, features, and advantages of the invention will become more apparent from the following detailed description, which proceeds with reference to the accompanying figures.

### BRIEF DESCRIPTION OF THE DRAWINGS

FIG. 1A shows a perspective view of a gurney with gurney attachment rods, in accord with aspects of the present disclosure.

FIG. 1B shows a plan view of the gurney with the gurney attachment rods in FIG. 1A, in accord with aspects of the present disclosure.

FIG. 2A shows a perspective view of the gurney with an arch positioning device, along with one degree of motion of the arch positioning device along the longitudinal axis of the gurney, in accord with aspects of the present disclosure.

FIG. 2B shows a perspective view of the gurney with the arch positioning device of FIG. 2A, and another degree of motion of the arch positioning device, rotating along the perpendicular axis of the gurney, in accord with aspects of the present disclosure.

FIG. 3A shows a perspective view of the arch positioning device and another degree of motion demonstrating unlocking one side of the arch and the vertical translation of the unlocked side along the pivot point of the contralateral attachment, in accord with aspects of the present disclosure.

FIG. 3B shows a side view of the arch positioning device and the other degree of motion in FIG. 3A, in accord with aspects of the present disclosure.

FIG. 3C shows a perspective view of the gurney and the arch positioning device, with another degree of motion of the positioning device demonstrating an unlocked arch pivoting on its remaining attachment and rotating orthogonally from the transverse to the longitudinal orientation, in accord with aspects of the present disclosure.

FIG. 3D shows a top view of the gurney and the arch positioning device, with the other degree of motion of the positioning device in FIG. 3C, in accord with aspects of the present disclosure.

FIG. 4A shows the arch positioning device at an oblique angle relative to the long axis of the gurney, in accord with aspects of the present disclosure.

FIG. 4B shows the arch positioning device at a perpendicular angle relative to the long axis of the gurney, in accord with aspects of the present disclosure.

FIG. 5 shows a side view of the arch positioning device and a transducer attachment, along with a degree of motion of the transducer attachment for translation of the transducer along the length of the arch, in accord with aspects of the present disclosure.

FIG. 6 shows a side view of the arch positioning device and the transducer attachment of FIG. 5, along with another degree of motion of the transducer attachment, in accord with aspects of the present disclosure.

FIG. 7 shows a side view of the arch positioning device and the transducer attachment of FIG. 5, along with another degree of motion of the transducer attachment demonstrating extension of the transducer radially away from the arch, in accord with aspects of the present disclosure.

FIG. 8 shows a control device for a positioning device, in accord with aspects of the present disclosure.

FIG. 9A shows a perspective view of an arm positioning device, in accord with aspects of the present disclosure.

FIG. 9B shows a side view of the arm positioning device of FIG. 9A, in accord with aspects of the present disclosure.

FIG. 10A shows a side view of the arm positioning device positioned relative to a gurney, along the long axis of the gurney, in accord with aspects of the present disclosure.

FIG. 10B shows a front view of the arm positioning device and the gurney, along the short axis of the gurney, in accord with aspects of the present disclosure.

FIG. 11 shows a plan view of the arm positioning device and an ultrasound machine in relationship to a sonographer, in accord with aspects of the present disclosure.

FIG. 12 is a schematic diagram of one embodiment of an arm positioning device, illustrating a ceiling mounted device.

FIG. 13 is a schematic diagram of an alternative embodiment of a ceiling mounted arm positioning device.

FIG. 14 is a schematic perspective diagram of a ceiling mounted, arm positioning device comprising a track mounted on a ceiling along which the device can be moved relative to a subject.

FIG. 15 is a schematic perspective diagram of a ceiling mounted arm positioning device track comprising plural ball and socket joints.

FIG. 16 is a schematic perspective diagram of an exemplary arm positioning device comprising a foundation, a plurality of arm segments and an instrument assembly and illustrating relative degrees of freedom for individual components.

FIG. 17 is a schematic perspective diagram of an exemplary instrument assembly.

FIG. 18 is a schematic perspective diagram of an alternative instrument assembly comprising a plurality of rotational joints and brackets.

FIG. 19 is a schematic perspective diagram of an alternative instrument assembly comprising a delta swivel mechanism.

FIG. 20 is a schematic perspective diagram of an alternative instrument assembly comprising plural ball and socket joints to facilitate effective movement of a medical instrument, such as an ultrasound transducer, relative to a subject.

FIG. 21 is a schematic perspective diagram of an alternative instrument assembly comprising a clamp, such as a threaded or spring-biased clamp, to grip a medical instrument, such as an ultrasound transducer.

FIG. 22 is a schematic diagram of an alternative instrument assembly comprising straps, such as Velcro® straps, to hold a medical instrument, such as an ultrasound transducer.

FIG. 23 is a schematic perspective diagram of an alternative instrument assembly comprising pliable bands and ratcheting straps to hold a medical instrument, such as an ultrasound transducer.

FIG. 24 illustrates different exemplary ultrasound probes.

FIG. 25 is a schematic, cross-sectional diagram of an exemplary pneumatic locking mechanism illustrating the mechanism in a locked configuration.

FIG. 26 is a schematic, cross-sectional diagram of the exemplary pneumatic locking mechanism of FIG. 25 illustrating the mechanism in an unlocked configuration.

FIG. 27 is a schematic perspective diagram of a wrap-spring brake mechanism comprising a single spring.

FIG. 28 is a schematic, cross-sectional diagram of a wrap-spring brake mechanism comprising two springs spiraling in different directions.

FIG. 29 is a schematic diagram of a fifth-link locking mechanism, illustrating how the mechanism attaches to a system comprising a four-bar linkage.

FIG. 30 is a schematic cut-away diagram of a sliding lock mechanism suitable for use as a fifth-link locking mechanism, illustrating the relative movement of the members of the locking mechanism.

FIG. 31 is an electrical circuit diagram, illustrating an exemplary control circuit for the locking mechanisms.

FIG. 32 is a schematic diagram of an alternative embodiment of the arm positioning device, illustrating 11 degrees of freedom.

FIG. 33 is a schematic diagram of an alternative embodiment of the arm positioning device, illustrating 6 degrees of freedom.

FIG. 34 is a schematic perspective diagram of an alternative instrument assembly comprising a hexapod assembly to facilitate effective movement of a medical instrument, such as an ultrasound transducer, relative to a subject.

### DETAILED DESCRIPTION

While the device, and method for using the device, discussed herein are susceptible to various modifications and alternative forms, specific exemplary embodiments are disclosed with reference to the drawings and will be described in detail herein. A person of ordinary skill in the art will understand that the description is not intended to be limited to the particular exemplary disclosed embodiments. Rather, the description covers all modifications, equivalents, and alternatives falling within the spirit and scope of the present disclosure and as defined by the appended claims.

For purposes of the present detailed description, the singular includes the plural and vice versa (unless specifically disclaimed); the word “or” shall be both conjunctive and disjunctive; the word “all” means “any and all”; the word “any” means “any and all”; and the word “including” means “including without limitation.” Additionally, the singular terms “a,” “an,” and “the” include plural referents unless context clearly indicates otherwise.

Embodiments of the disclosed positioning device, and a method of using the same, provide benefits to a user, such as to: (1) facilitate movement of a medical instrument, such as an ultrasound transducer; and/or (2) minimize or eliminate the compressive force that a user may generate during a procedure, for example, the force that a sonographer typically generates during an ultrasound examination. The positioning devices provide a solid support structure from which to extend an instrument, such as a transducer, particularly an ultrasound transducer, and/or to apply compressive force against the patient during an examination or procedure. In some embodiments, the compressive force generated by the positioning device allows a sonographer to manipulate the ultrasound transducer such that an examination can occur without requiring the sonographer to generate all, or potentially any, of the compressive force, while still enabling accurate and detailed examinations. In addition to applying compressive force, the positioning device provides other assistance during ultrasound examinations or other types of examinations and/or procedures, which alleviate the burden experienced by the sonographer to maintain a rigid position with the transducer, often while applying prolonged pressure force, and offers additional assistance previously not possible. For example, disclosed embodiments are suitable to maintain an instrument in a position while allowing the user/operator to be free to perform other actions or procedures without losing pressure or position on the patient. The user is potentially able to use both hands for additional tasks, thereby allows for greater productivity and independence when performing procedures and examinations. Additionally, certain embodiments may facilitate applying pressure for improved imaging in otherwise hard to reach angles and locations.

### A. Arch Positioning Device

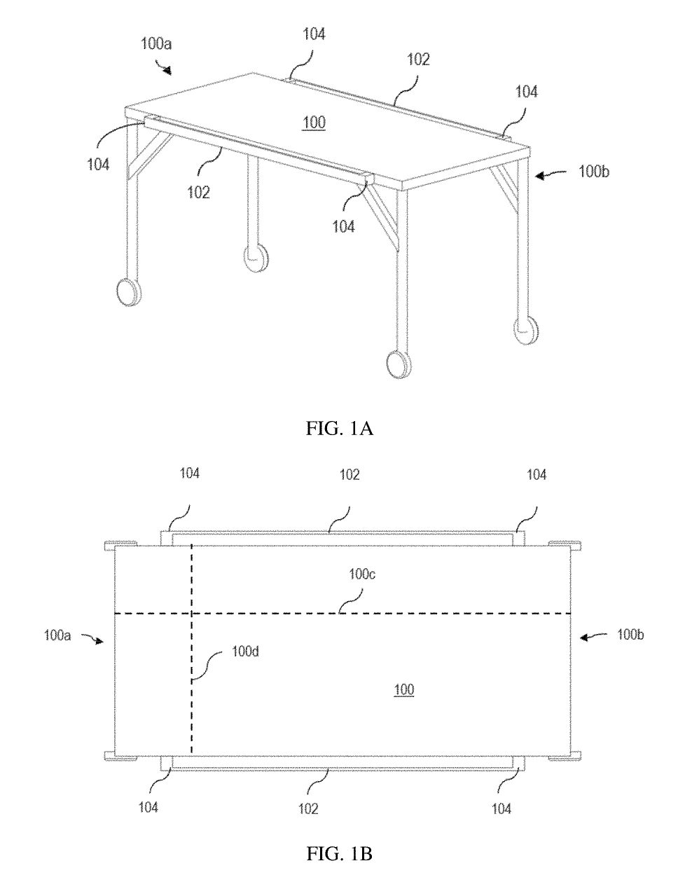

Referring to FIGS. 1A and 1B, FIG. 1A shows a perspective view of a gurney 100 with gurney attachment rods 102, and FIG. 1B shows a plan view of the gurney 100 with the gurney attachment rods 102, in accord with aspects of the present disclosure. The gurney attachment rods 102 are rods placed on both sides of the gurney 100. The gurney attachment rods 102 can have various shapes, such as, for example circular, square, triangular, etc. in cross-section. The gurney attachment rods 102 serve as attachment points for a positioning device to the gurney 100. Each gurney attachment rod 102 includes attachment points 104 to the gurney 100, likely manifesting as, but not limited to, one at a superior position 100a of the gurney 100 and one at an inferior position 100b of the gurney 100. The attachment points 104 can be at the most superior position 100a and the most inferior position 100b relative to the gurney 100 to reduce interference with other components of a positioning device along the long axis 100c of the gurney 100 and provide the greatest range of motion for the other components.

The attachment points 104 for the gurney attachment rods 102 can be permanent or removable from gurney 100. In the case of removable attachment points, each attachment point 104 includes a mechanism to couple to and decouple from the gurney 100. The mechanism can be, for example, a vice or a clamp, such as a quick-lock clamp, or any other mechanical or electromechanical mechanism.

The gurney attachment rods 102 extend off of the sides of the gurney 100 a desired distance to allow sliding access between the gurney attachment rods 102 and the gurney 100 to provide adequate room for a positioning device to slide up and down the gurney attachment rods 102 while also not being obtrusive; by way of example, and without limitation, the distance can be 1 to 15 centimeters.

Depending on the configuration of the gurney 100, however, alternatively the positioning device may not include the gurney attachment rods 102, or a positioning device may be separate from the gurney attachment rods 102. Rather, for example, the gurney 100 may include rods already affixed to and integral with the gurney 100. The positioning device may attach directly to the gurney 100 by way of the included rods, rather than to the gurney 100 by way of the gurney attachment rods 102.

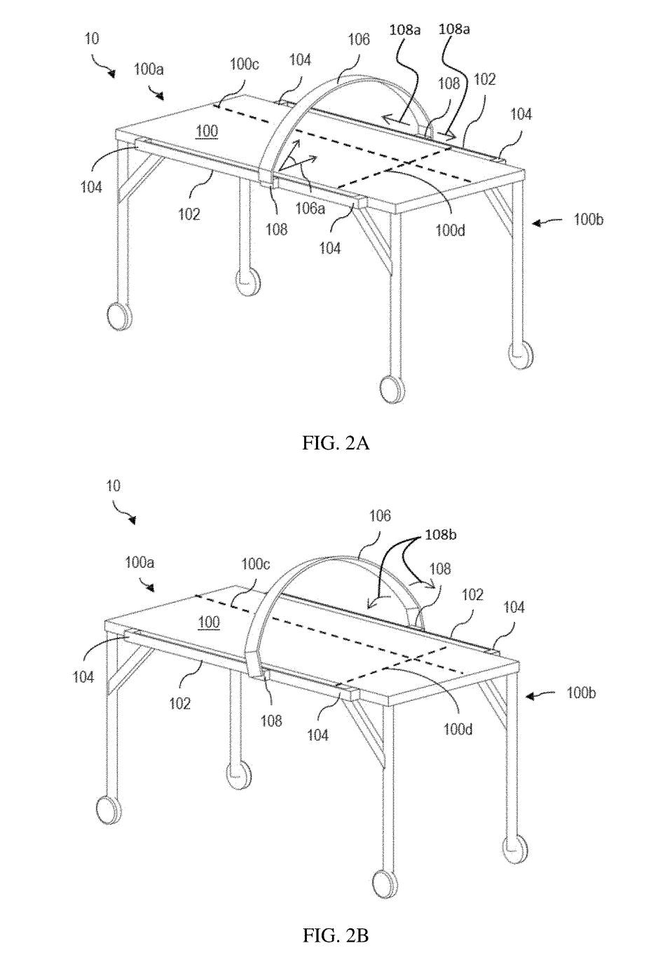

Referring to FIG. 2A, an arch positioning device 10 is shown, in accord with aspects of the present disclosure. The arch pressure application assist device 10 includes an arch 106. Although illustrated and described as an arch 106, the structure of the arch 106 is not limited in function to an arch shape but can embody any supporting structure regardless of shape. The arch 106 is a supported rounded frame (or semi-ovoid arc) with a length that extends across the short axis 100d of the gurney 100 and an arc angle 106a that accommodates the body habitus of varying patients. Arc angle herein is defined as its mathematical analogue which represents the angle an arc makes at the center of the circle of which it is a part. The arc angle 106a can vary without departing from the spirit and scope of the present disclosure, such as being, for example, from greater than zero to 180°, such as from 20° to 90°, or from 30° to 60°. Modifying the arc angle allows for quick and simple changes to the relative position of the instrument, such as a transducer, from the top of the gurney allowing for convenient modification for patients of varying sizes from neonatal to morbidly obese. In some embodiments, the arc angle of the arch as well as its elevation from the top of the gurney are modifiable allowing for simple adaptation of the arch to closely approximate the girth of a patient's body.

The arch 106 includes two connection points 108 that attach the arch 106 to the gurney attachment rods 102 discussed above. The connection points 108 are located at substantially the ends of the arch 106. However, the connection points 108 may alternatively be located along the length of the arch 106 (i.e., not at the ends) depending on the length of the arch 106 and the position of the arch 106 relative to the gurney 100. The connection points 108 can be various mechanisms that allow the arch 106 to couple to and decouple from the gurney attachment rods 102. By way of example, and without limitation, the connection points 108 can be clamps that attach to the gurney attachment rods 102.

As shown in FIG. 2A, the connection points 108 provide one or more degrees of freedom of motion to adjust the arch 106 relative to the patient and/or gurney 100. Both connection points 108 may have the same degrees of freedom. Alternatively, one of the connection points 108 may have more degrees of freedom than the other. The connection points 108 allow the arch 106 to move along the length of the long axis 100c of the gurney 100, as shown by the arrows 108a.

Referring to FIG. 2B, the connection points 108 also enable the arch 106 to tilt superiorly towards the superior position 100a of the gurney 100 and inferiorly towards the inferior position 100b of the gurney 100, as shown by arrows 108b.

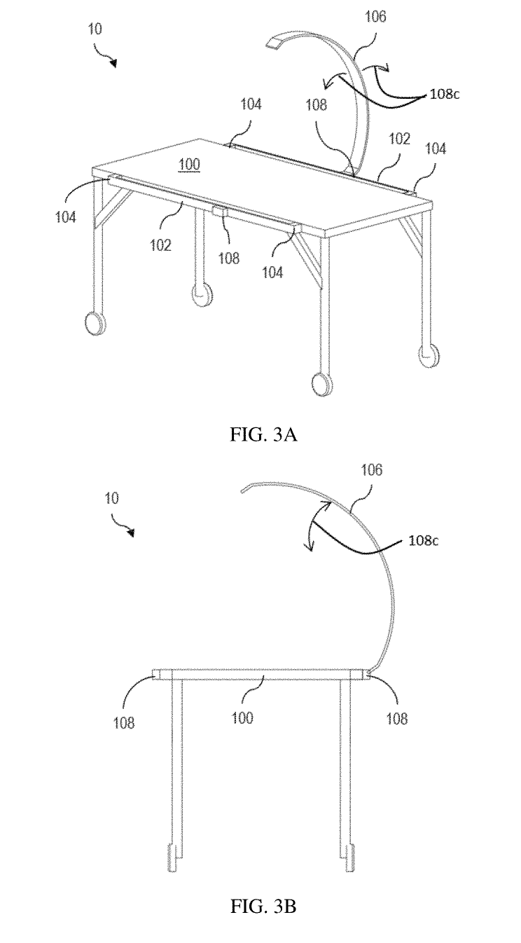

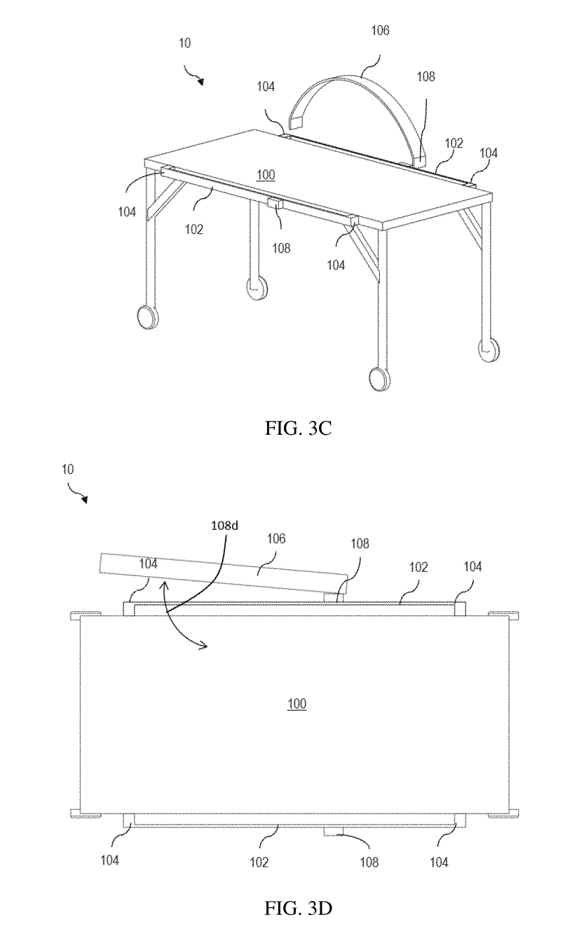

Referring to FIGS. 3A and 3B, the connection point 108 can include a degree of freedom that allows the arch 106 to rotate upward, as shown by arrows 108c, between 60° and 120° as desired by the sonographer, away from the gurney 100, when the arch 106 is detached from the opposite connection point 108.

Referring to FIGS. 3C and 3D, the connection point 108 may, in addition, or in the alternative, have a degree of freedom that allows the arch 106 to rotate horizontally along the plane of the gurney 100, as illustrated by arrows 108d. One or both the degrees of freedom discussed with respect to FIGS. 3A-3D enable a patient to access the gurney 100 without interference from the arch 106, if the arch cannot simply be moved to an extreme position along the length of the gurney to allow simple embarking or disembarking of the patient.

The connection point 108 may be permanently attached to the arch 106 and then lowered and clamped onto the gurney attachment rod 102 (or the gurney 100) when in use. Alternatively, the connection point 108 may be permanently attached to the gurney attachment rod 102 (or the gurney 100) and the arch 106 can be lowered and locked onto the connection point 108 when in use. The one or both connection points 108 can include a button or a latch to allow for locking and unlocking of the arch 106 to the connection point 108 or the gurney attachment rod 102 to allow the sonographer to open and close the arch 106 for patient access.

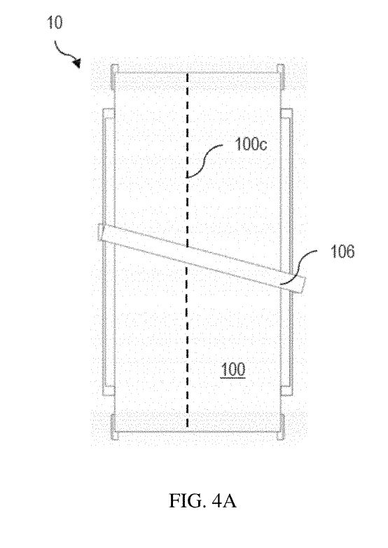

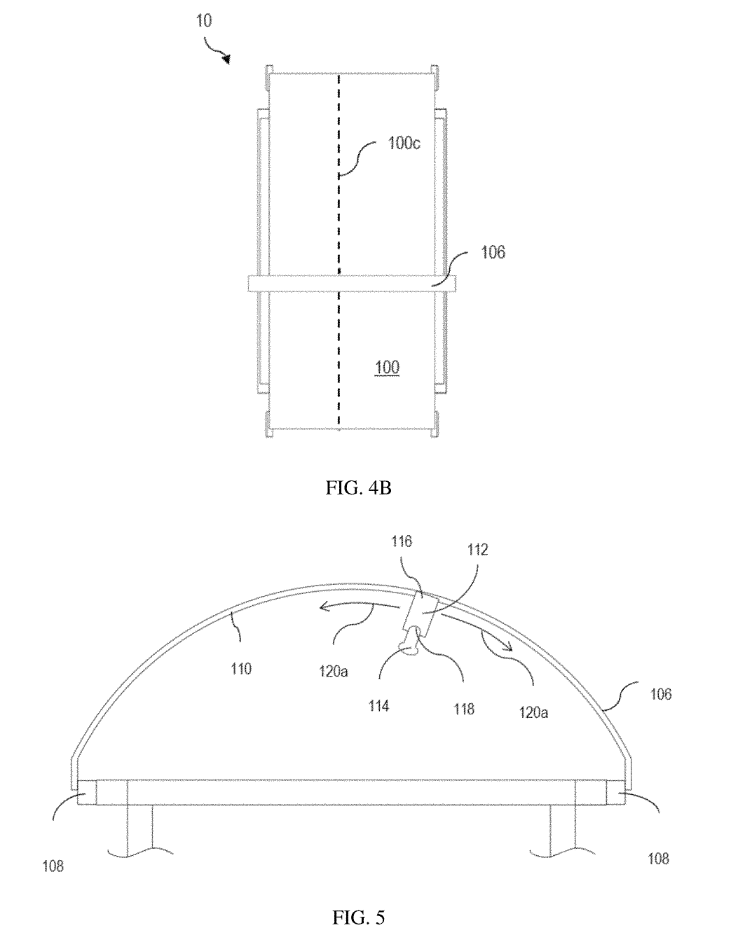

Referring to FIGS. 4A and 4B, when connected to the gurney attachments rods 102, the arch 106 maintains a fixed perpendicular orientation relative to the gurney attachment rods 102 as the arch 106 slides along the long axis 100c of the gurney 100. Alternatively, or in addition, the connection points 108 may be configured to allow rotation of the arch 106 at the connection points 108, thus enabling the length of the arch 106 to be angled relative to the long axis 100c of the gurney 100. For example, FIG. 4A shows the arch 106 oblique to the long axis 100c of the gurney 100, and FIG. 4B shows the arch 106 perpendicular to the long axis 100c of the gurney 100. The arch 106 being configured to be oblique to the long axis 100c allows for further manipulation of the arch 106 during an examination or procedure, if, for example, the arch 106 interferes with the positioning of the ultrasound transducer 114 (discussed below). Such a configuration relies on the arch 106 to be amenable to accommodate the greater distance required for the arch 106 to span across the gurney 100. In this regard, the arch 106 can be rigid or inflexible when exposed to normal loads during manipulation and movement before, during, and/or after an examination. Alternatively, the arch 106 can be semi-flexible to allow the arch 106 to change its shape before, during, and/or after an examination, such as becoming angled to the long axis 100c of the gurney 100 when exposed to normal loads during use. The arch 106 may be formed of metal, such as stainless steel, titanium, etc., or a metal alloy in the example of a rigid arch, or may be formed of a hard plastic or rubber in the example of a semi-flexible arch.

Referring to FIG. 5, the arch 106 also includes a traversable component, herein referred to as the track 110, that runs substantially the entire length of the arch 106. The track 110 can run along the inner circumference of the arch. Alternatively, or in addition (in the case of multiple tracks 110), the track 110 can run along the side and/or external circumference of the arch 106. The track 110 accommodates a transducer attachment 112. The track 110 and the instrument attachment 112 allow for the positioning of the instrument, such as an ultrasound transducer 114 along the length of the arch 106. The instrument attachment 112 can be configured to accept existing ultrasound transducers for a variety of examinations that may require ultrasound imaging. The instrument attachment 112 includes an attachment site 116 that connects the instrument attachment 112 to the track 110, and an instrument holder 118 that connects the transducer 114 to the instrument attachment 112. The instrument holder 118 can accommodate any transducer placed within it and hold it securely without interfering with the natural use of the sonographer. The instrument attachment 112 is configured to provide for one or more degrees of freedom of motion. As shown in FIG. 5, one degree of freedom, as represented by the arrows 120a, allows the instrument attachment 112 to travel along the length of the arch 106 (e.g., along the track 110) allowing for complete, or substantially complete, transverse coverage of the patient. The instrument attachment 112 has a braking mechanism at its attachment to the arch 106 or track 110 allowing it to be dampened and locked in its position.

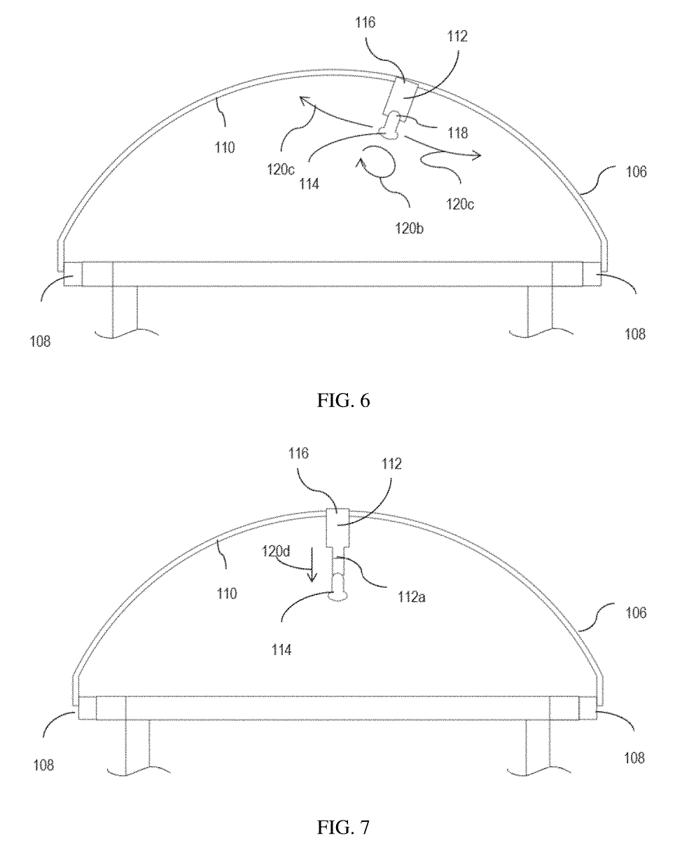

Referring to FIG. 6, according to another degree of freedom, as represented by the circular arrow 120b, the instrument attachment 112 is configured to provide substantially 360° of rotation. Thus, with the transducer 114 connected to the instrument attachment 112, the transducer 114 can be rotated substantially 360°. As further shown in FIG. 6, according to another degree of freedom, as represented by the arrows 120c, the instrument attachment 112 is configured to provide tilt angling up to 80° in any direction relative to the orthogonal orientation of the instrument attachment tangent to the arch, i.e. its diameter. The ability for the transducer 114 to rotate and tilt allows for a sonographer to position the ultrasound transducer 114 as needed to acquire signal and transverse images and enables the transducer 114 to be tilted in any direction for fine tuning of images. Although described as allowing for angles of up to 80° in all directions, alternatively, the ranges of motion can be configured to be less than 80° to limit the range of motion of the arch positioning device 10, such as for safety and/or manufacturing concerns.

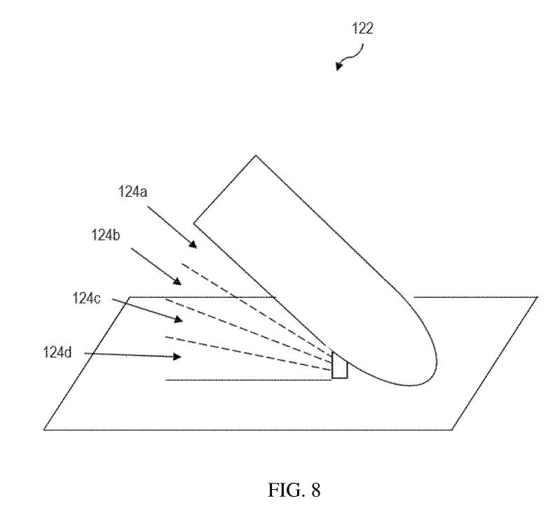

Referring to FIG. 7, for pressure assistance, the instrument attachment 112 is configured to extend the instrument, such as transducer 114, towards the patient to apply pressure. The instrument attachment 112 can include various mechanisms that extend the instrument towards the patient, such as, for example, an electrical, pneumatic, and/or hydraulic actuator, a pneumatic or hydraulic cylinder, or an electrical motor that controls the extension of a rod, or similar element, included within the instrument attachment 112. The mechanism, such as a linear coil actuator, can generate fine movements of the instrument to allow for fine positioning during an examination and fine control of the pressure. As shown in FIG. 7, the instrument attachment 112 includes a linear coil actuator 112a that extends the instrument linearly in a direction defined by the long axis of the linear coil actuator 112a, represented by the arrow 120d. Accordingly, a sonographer can position the transducer 114 based on the rotational and tilting degrees of motion described above, and then apply pressure by extending the transducer 114 by activating the linear coil actuator 112a. The linear coil actuator 112a, or similar mechanism, is configured to provide pressure that eliminates the need for pressure supplied from a sonographer during an examination. Alternatively, the linear coil actuator 112a, or similar mechanism, is configured to apply an amount of pressure that supplements the pressure provided by the sonographer, such as to eliminate a majority of the pressure from the sonographer required for an examination.

The instrument attachment 112 can include an additional degree of freedom during extension of the linear coil actuator 112a (or similar device) beyond just the linear direction 120d. For example, the instrument attachment 112 can be configured to rotate the linear coil actuator 112a as the linear coil actuator 112a extends towards the patient. The rotation can provide additional assistance to the sonographer, such as additional manipulation assistance, or other assistance as described below, beyond the field of ultrasound examinations.

The arch positioning device 10 includes various cables (not shown) that lead from one element, such as the arch 106, the instrument attachment 112, etc., to a control device to control the operation of the arch positioning device 10. The track 110 can be configured to allow one or more cables from the transducer 114 (if not wireless) to run along the length of the arch 106 so as to stay off the patient and out of the sonographer's examination field.

Referring to FIG. 8, the arch positioning device 10 further includes a control device. The control device can be various different types of devices, e.g. pedal, trigger on transducer attachment, lever or button, that allow a user to control the arch positioning device 10 during an examination. Because the user may need to manipulate the arch positioning device 10 using his/her hands, the control device can be, for example, a pedal 122 that is operated by the user using a foot. The control device may be an analog device, a digital device, or a combination thereof. Accordingly, the user can operate the pedal 122 with her foot while manipulating the other components of the arch positioning device 10 with her hands.

The arch positioning device 10 may be configured and operated according to a semi-passive configuration or an active configuration. According to a semi-passive configuration, the pedal 122, or other control device, allows a user to control the dampening, locking, and pressure activation of the arch positioning device 10. The pedal 122, or other control device, provides a gradient dampening of motion and increased pressure depending on the degree of pedal depression. By way of example, and without limitation, the pedal 122 can include different zones for each degree of freedom of the arch positioning device 10. According to the configuration discussed above, the pedal 122 includes four zones along the range of pedal depression. Each zone can have the same range of depression, or different ranges of depression, depending on, for example, the sensitivity required for the associated degree of freedom.

Referring back to FIG. 8, each zone can be represented by a portion of the entire range of depression, such as a quarter of the entire range. As the pedal 122 is depressed into the first zone, moving or tilting of the arch 106 along the gurney attachment rods 102 along the long axis 100c of the gurney 100, as shown by arrows 108a and 108b in FIGS. 2A and 2B, respectively, is dampened. As the pedal 122 is depressed within the first zone 124a, the ability to move the arch 106 in the directions of arrows 108a and 108b is further limited or dampened. When the pedal 122 is depressed to the limit of the first zone 124a, movement along arrows 108a and 108b locks and the arch 106 is no longer able to move in those directions or motions. Each of these transitions resulting from increased pedal depression can include a haptic or tactile transition cue, e.g. a tangible click or ridge, allowing the user to know when different dampening/locking ranges are being traversed.

As the pedal 122 is depressed into the second zone 124b, dampening begins for the second degree of freedom. According to the configuration discussed above, the second degree of freedom may be the motion of the transducer attachment 112 along the track 110. As the pedal 122 is depressed within the second zone 124b, the ability to move the instrument attachment 112 within the second degree of freedom is further limited or dampened. When the pedal 122 is depressed to the limit of the second zone 124b, the second degree of freedom locks and the instrument attachment 112 is no longer able to move within the second degree of freedom.

As the pedal 122 is depressed into the third zone 124c, dampening begins for another degree of freedom. Although discussed above with respect to one zone representing one degree of freedom, alternatively, one zone may represent multiple degrees of freedom. For example, the third zone 124c may correspond to the two degrees of freedom (e.g., third and fourth degrees of freedom) associated with the instrument attachment 112 allowing the instrument, such as transducer 114, to rotate and tilt. As the pedal 122 is depressed within the third zone 124c, the ability to rotate and tilt the transducer 114 relative to the instrument attachment 112 is further limited or dampened. When the pedal 122 is depressed to the limit of the third zone 124c, the third and fourth degrees of freedom lock and the transducer 114 is no longer able to move relative to the instrument attachment 112 within the third and fourth degrees of freedom. Alternatively, rather than locking the third and fourth degrees of freedom upon reaching the limit of the third zone 124c, the pedal 122 may instead dampen the third and fourth degrees of freedom but still allow for fine movements of the transducer 114. Such fine movements allow for the sonographer to manipulate the transducer 114 as needed for fine tuning images during an examination. These various zones can be customized to be tailored to specific examinations that require different patterns of dampening/locking, as well as ad hoc by the user.

One or more of the zones of the pedal 122 that control dampening can include a bypass option for the user to quickly enable a specific zone to lock completely during instances when the user wishes to take her hand(s) off of a particular site on the patient but still maintain the correct position and pressure of the instrument. Alternatively, or in addition, one or more zones of the pedal 122 that control dampening can include a quick release that releases the lock on the degree of freedom, allowing the user to move the element associated with the degree of freedom without dampening and/or locking from the pedal 122. These controls can be located in a variety of ergonomically convenient locations, for instance on the user dashboard on the unit or can be combined into a series of buttons or switches on the instrument attachment.

The fourth (or final) zone 124d controls a dynamic and gradient amount of pressure from the instrument attachment 112 for applying pressure at the site of interest of a patient. As the user depresses the pedal 122, the depression causes the instrument, by way of the linear coil actuator 112a, to extend linearly towards the patient, applying a greater amount of pressure once the instrument contacts the patient. The degree of depression of the pedal 122 determines the degree of pressure applied through the instrument. In some embodiments, the amount of pressure applied is directly proportional to the degree of pressure applied to the control device. The pedal 122 may include a set maximum amount of pressure that can be applied, such as, for example, 15 foot-pounds of maximum pressure. Accordingly, the positioning device will not exceed this limit, or will automatically reduce the pressure if the maximum pressure is temporarily exceeded. The positioning device may also automatically reduce the pressure if the maximum pressure is reached. Accordingly, in some configurations, depression of the pedal 122 beyond a set point does not increase the pressure at the patient to prevent injury to the patient. Again, each of the prior degrees of freedom can be locked allowing the user to keep the instrument in a stable position and the pedal used only to alter the degree of pressure, or the degree of pressure can also be locked allowing for a maintained specific view with a precise pressure and orientation.

As an alternative or addition to the control of the pressure based on the amount of depression of the pedal 122, the pedal 122 may be connected to a computer that allows the user to enter a specific amount of pressure to apply using the arch positioning device 10. The pressure can be determined based on the region of the procedure to be performed, the gender of the patient, the weight and/or age (e.g., pediatric versus adult) of the patient, the depth of the target for the procedure, among various parameters. With a computer connected to the arch positioning device 10, the computer can store information pertaining to the examination and/or procedure. Such information can include, for example, the positioning of the arch positioning device 10 relative to the patient and/or gurney, pressure applied to the patient, anatomy of the patient that was the focus of an examination, etc. Thus, and by way of example, the arch positioning device 10 can be repositioned to a previous position based on the position being stored in the computer. The computer can provide additional functionality, such as being compatible with electronic medical records systems and/or hospital information technologies through wired or wireless connectivity, such as Wi-Fi, Bluetooth®, or similar wireless networking protocols.

As an alternative, the arch positioning device 10 may include two pedals. One pedal may control the dampening and locking of the arch positioning device 10 and the other pedal may control the pressure provided by the arch positioning device 10. Alternatively, the arch positioning device 10 can include other forms of control devices, such as knobs, buttons, dials, etc.

During an examination without the arch positioning device 10, the user is able to feel the amount of force that is being applied to the patient because the user is applying the pressure. Accordingly, the instrument attachment 112 can include a force feedback mechanism that allows the user to feel an indication of the amount of force that is being applied to the patient. During an examination, the instrument attachment 112 can provide a tactile response to the user that allows the user to determine the amount of pressure that is being applied to the patient, such as through a reactive resistance as the control unit is activated, for example, as a pedal is depressed. This allows the user to feel the amount of pressure being applied to minimize the risk of injury to the patient. As an alternative, or in addition to the tactile feedback response, the arch positioning device 10 may provide a visual and/or auditory response indicating the amount of pressure that is being applied to the patient. The user may then adjust the amount of pressure based on the visual and/or auditory response. By way of example, and without limitation, the instrument attachment 112 can include an indicator light that lights up when the instrument attachment 112 reaches a set or maximum pressure or provide a visual array that increases in intensity or number of lights as pressure increases. As an alternative, or in addition to the tactile or visual/auditory feedback response, the pressure pedal itself can increase its resistance as more pressure is applied and in direct proportion to the reactive resistance from the patient, allowing the user to palpate directly the quantity of pressure applied through the pedal itself.

As mentioned above, alternatively or in addition, the arch positioning device 10 can have an active configuration. The active configuration allows a user to actively control the positioning of the arch positioning device 10 with the control device, such as the pedal 122, rather than manually. The arch positioning device 10 can include one or more motors for each degree of freedom discussed above that power movement of the arch positioning device 10 with respect to the particular degree of freedom. Accordingly, under the active configuration, a user can control the position of the arch positioning device 10 using a separate set of pedals or joysticks allowing for individual control of the respective degrees of freedom. Further, the active control of the position of the arch positioning device 10 allows for the application of additional pressure, beyond the pressure provided by the instrument attachment 112, or additional manipulation of the instrument relative to the patient, by actively moving the arch positioning device 10. For example, the user can tilt the arch 106 to provide additional pressure. In an active configuration, the pedal 122 (or other control device, e.g. joystick) can be remote from the arch positioning device 10 such that the user can control the arch positioning device 10 from a different room.

The arch positioning device 10 can include additional components without departing from the spirit and scope of the present concepts. For example, the arch positioning device 10 can include a display (not shown) that allows a user to view the results of the examination with the instrument to maximize comfort of the user during an examination (e.g., reduce neck strain, back strain, etc. from having to adjust to both view the display and view the instrument). The display can include various information, such as pressure force, angles of the elements (e.g., arch 106), which elements are locked, etc. Alternatively, such a display can be integrated into an existing ultrasound system, and/or such information can be presented on the display of an existing ultrasound system.

### B. Active or Semi-Passive Arm Positioning Device

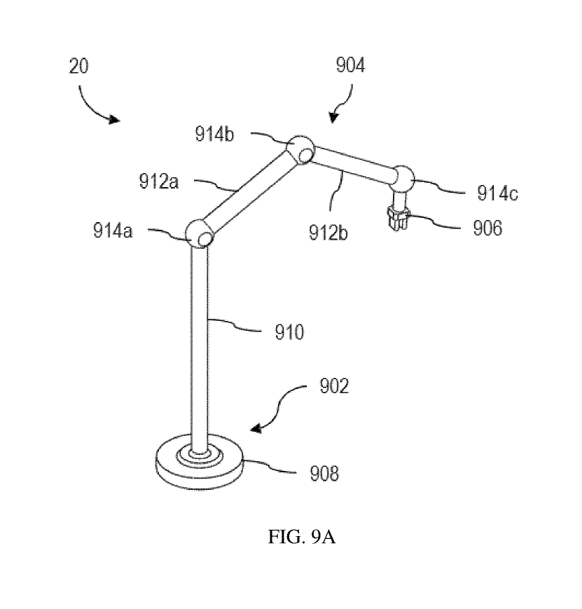

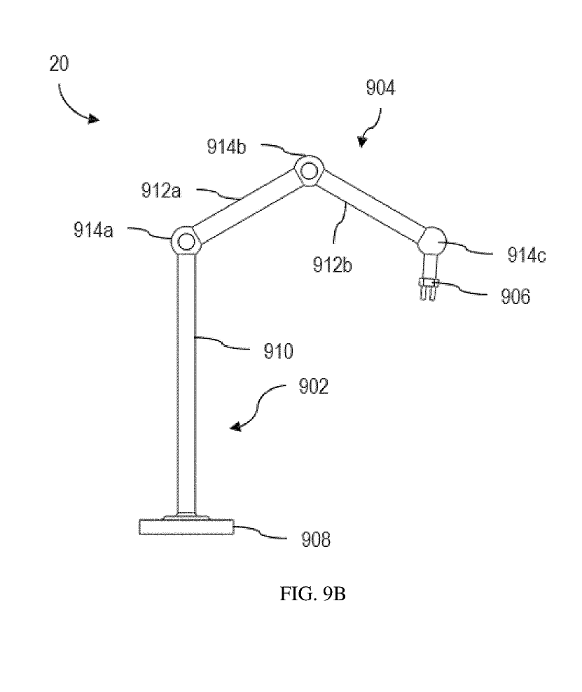

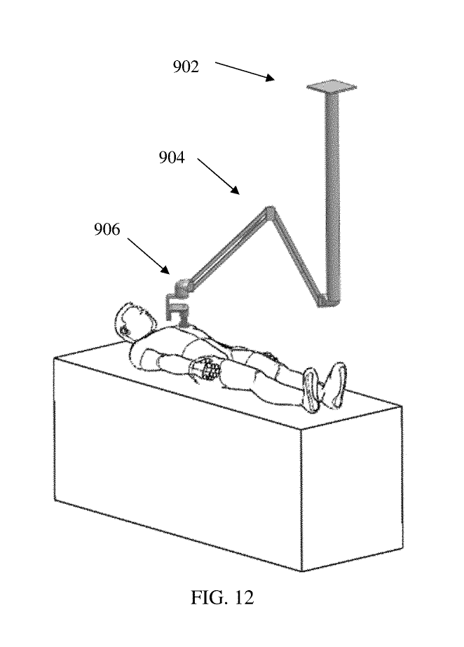

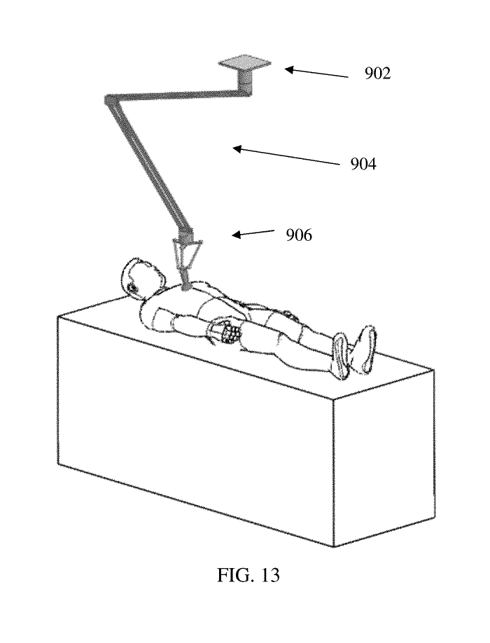

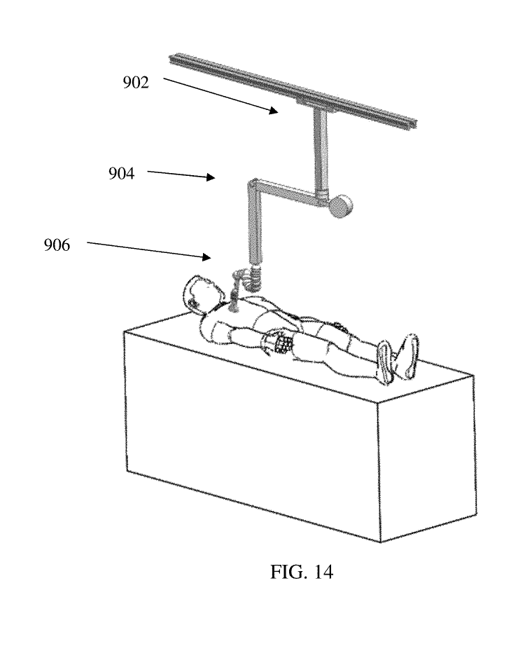

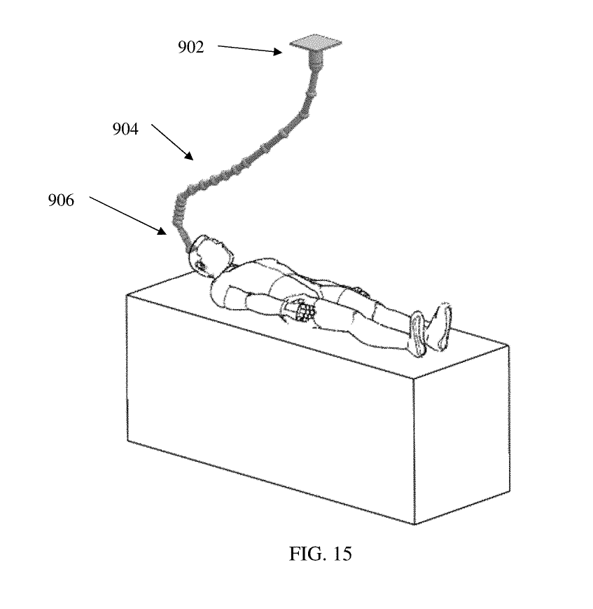

An alternative embodiment of a positioning device is an arm positioning device. FIGS. 9A and 9B illustrate exemplary floor-mounted embodiments, and FIGS. 12-15 illustrate exemplary ceiling mounts. With reference to FIG. 12, one embodiment of a positioning device 20 comprises a foundation 902, an arm 904, and an instrument attachment 906.

The foundation 902 provides a stable and secure platform for the arm positioning device 20 to apply and/or maintain pressure to the patient. As shown in FIGS. 9A and 9B, the foundation 902 can be a permanent attachment to the floor. Alternatively, the foundation 902 can be an ultrasound machine, scaffold mounts on the ceiling or wall, a portion of the gurney (e.g., gurney attachment rods 102), or the like. Alternatively, the foundation 902 can be a temporary attachment to a gurney or other portable object. The foundation 902 may be one of various different forms and configurations, such as, without limitation, a fixed floor mount, a mobile floor mount, a ceiling mount, a wall mount, or a furniture attachment, such as for a desk, a cabinet, a gurney/bed, or to a medical instrument machine, such as an ultrasound machine. The type of foundation 902 may depend on, for example, the location of the arm positioning device 20, such as whether the room in which the arm positioning device 20 is located is a dedicated examination or ultrasound room or a surgical suite with existing ceiling mounted scaffolding, etc.

As shown in FIGS. 9A and 9B, the foundation 902 may be considered the first segment of the arm 904, or it may be considered a separate element from the arm 904. For example, the foundation 902 may include a base 908 and a stand 910 that extends up from the base 908. In such a configuration, the base 908 and the stand 910 may be immobile, such that the base 908 and the stand 910 cannot move relative to each other or relative to the patient. The base 908 and the stand 910 can be considered separate components of the arm positioning device 20 than the arm 904. Alternatively, the foundation 902 may instead be an attachment point at the end of the arm 904, such as in the base of an arm positioning device that attaches directly to the gurney 100 or to scaffold mounts. In which case, the foundation can be considered part of the arm 904. In either case, the foundation 902 provides support for the arm 904 at a location that will not interfere with the patient or the sonographer. The base 908 can include a slip-clutch in the event of an emergency to be able to move the arm positioning device 20.

The arm 904 may be unitary, or it may comprise plural segments and joints along its length from an initial segment or joint to a final joint connecting to the transducer attachment 906. As shown in FIGS. 9A and 9B, the arm positioning device 20 can include segments 912a, 912b and joints 914a-914c. The joints 914a-914c can be various types of joints, such as hinge joints, ball joints, etc. The joints 914a-914c along the arm can have one or more degrees of freedom of motion. By way of example, and without limitation, all of the joints, with the exception of the most distal joint 914c, may have a single or multiple degrees of freedom of motion. Each degree of freedom of the joints 914a-914c may be the same degree of freedom or a different degree of freedom. For example, joints 914a and 914b may allow for a tilting motion. The tilting motion allows the segment attached to the joint to tilt along the axis of the joint as well as rotate substantially 360° within the plane defined by the joint both immediately proximal to the joint 914a-914c as well as immediately distal to the joints (these representing further degrees of motion). The rotational motion allows the segment attached to the joint to rotate orthogonally 360° around the joint.

Although disclosed and shown as including three joints 914a-914c and two segments 912a, 912b, the arm 904 may have more or fewer joints and/or segments. For example, a greater number of joints and segments may allow for the arm 904 to conform to a greater number of positions, and a fewer number of joints may reduce manufacturing costs and complexity.

Wires on the arm 904 run along the length of the arm 904 and out to a control device, either contained within the arm components or immediately outside the arm components but isolated from external contact, thereby staying away from the patient and out of the examination or procedural field of the user or operator.

Movement of the arm positioning device 20 may be entirely passive, relying on a user to both move the arm 904 and support the arm 904 until dampening and locking with the control device, as described below. Alternatively, movement of the arm positioning device 20 can be semi-passive. Specifically, the joints 914a-914c may provide active resistance to movement under the force of gravity to prevent the segments 912a, 912b from moving under gravity. In such a configuration, the arm 904 does not move without the user's involvement. Specifically, the arm 904 can provide active assistance that is initiated and directed by the user moving the arm 904. Accordingly, a user can grasp the instrument attachment 906 or the distal-most segment 912b and move it to the desired location, and the movement assist will mechanically follow the intended path. In such a configuration, the joints 914a-914c provide immediate responsiveness to the user's active motions with little to no resistance and will hold their last positions when the user releases or halts movement. Servos or detectors within the joints also sense the direction, acceleration and velocity of their motion which is delivered to the central processor which then communicates back to those specific servos to assist with motion in that same direction and with the same, or slightly increased, acceleration and velocity, thereby providing active motion assistance. This is dynamically updated so that complex maneuvers can be communicated near-instantaneously to the various servos and joints to coordinate a fluid experience by the user so she will experience minimal to substantially no inertia from the mass of the arm.

The instrument attachment 906 extends from the most distal joint 914c on the arm 904. The instrument attachment 906 and the most distal joint 914c provide degrees of freedom of motion as discussed above with respect to the arch positioning device 10, enabling 360° rotation as well as 360° tilting of an instrument (e.g., transducer 114) connected to the instrument attachment 906. Similarly, the instrument attachment 906 provides pressure application assistance similar to the arch assist device 10. For example, the instrument attachment 906 can include a linear coil actuator, or other mechanism discussed above (not shown), that extends an attached instrument (not shown) linearly in a direction away from the instrument attachment 906. Accordingly, a user can position the instrument attachment 906 based on the rotational and tilting degrees of motion described above, and then apply pressure by activating the linear coil actuator within the instrument attachment 906.

Additionally, pressure application assistance need not only be limited to the linear direction of the axis of the instrument attachment along the course of the instrument. There may be circumstances during an examination where an oblique angle of pressure application may be useful, in which case pressure application assistance would be achieved by the user indicating on the control unit 918 an alternative pressure direction. This can be simulated by applying pressure using the mechanical joint actuators along the most recent direction of motion, instead of being limited to the linear actuator 112a. The system may record one or multiple, such as between 2 and up to at least 50, of the most recent directions of motion performed by the user and allow the user to select between them based on a visual display of the direction on a screen and/or through a tangible short motion of the arm to simulate the direction for the user to sample. When this alternative pressure angle is toggled, the user can choose which of the most recent motions is desired from the control unit and then apply pressure to an actuator to administer pressure at that new angle. Toggling off this feature of alternative angle of pressure application will return the system to the default angle of pressure application along the axis of the linear actuator 112a.

The arm device 20 can be controlled according to various configurations and mechanisms, such as those described above with respect to the arm positioning device 10. Accordingly, the arm positioning device 20 can be controlled by a pedal, such as the pedal 122 discussed above, or alternatively a trigger, button or level. For the arm positioning device 20, the pedal 122 functions essentially the same as in the arch positioning device 10. However, each of the different zones of the pedal 122 instead corresponds to a different joint (or joints) 914a-914c along the length of the arm 904. For example, the zones 124a-124d relate to the various joints of the arm 904, with the first zone 124a of the pedal 122 corresponding to the proximal-most joint 914a, the second zone 124b corresponding to the joint 914b, the third zone 124c corresponding to the third joint 914c, and the fourth zone 124d corresponding to the pressure provided by the instrument attachment 906. Accordingly, the pedal 122 provides the same dampening, locking, and pressure assistance as described above relative to the arch positioning device 10. Alternatively, the control unit can have a control that reverses the pedal and dampening/locking relationship so that the arm may be set to be locked at all the joints and pedal pressure causes a progressive release of the joints allowing movement. In this setting, once in position, the arm can be locked again by releasing the pedal. In this setting pressure can be performed by a second control unit. In configurations with a greater number of joints, the pedal 122 can include a greater number of zones, or controls for certain zones may be offloaded to another control device, such as another pedal or similar mechanism that can be controlled with the user's feet, hands, or even under voice control. Alternatively, all functions of the arch and arm positioning devices 10 and 20 can be voice-controlled to free up both the user's hands and feet during an examination procedure. For instance, pressure can be directed through a voice command allowing for hands free pressure and guided to a specific pressure by a preselected quantity or a verbal instructed value.

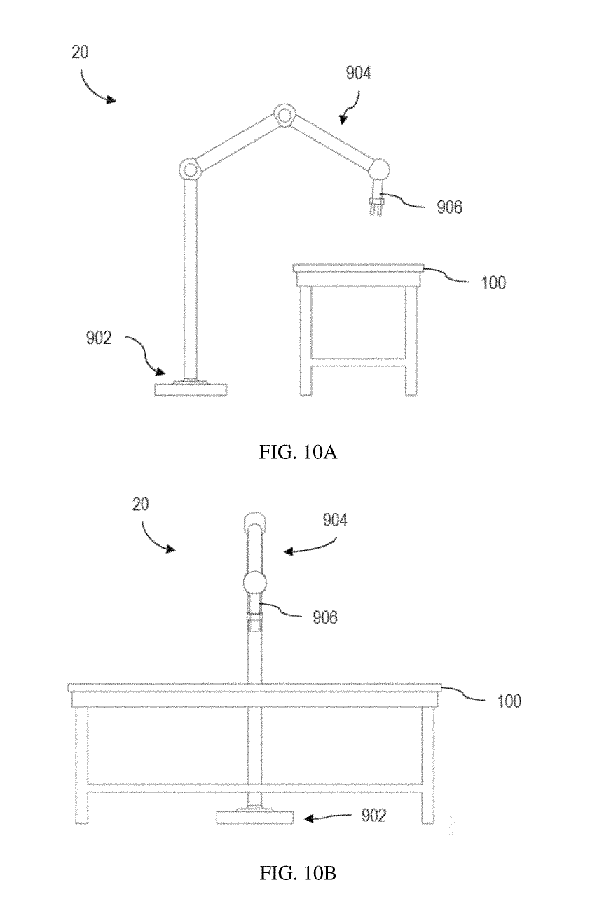

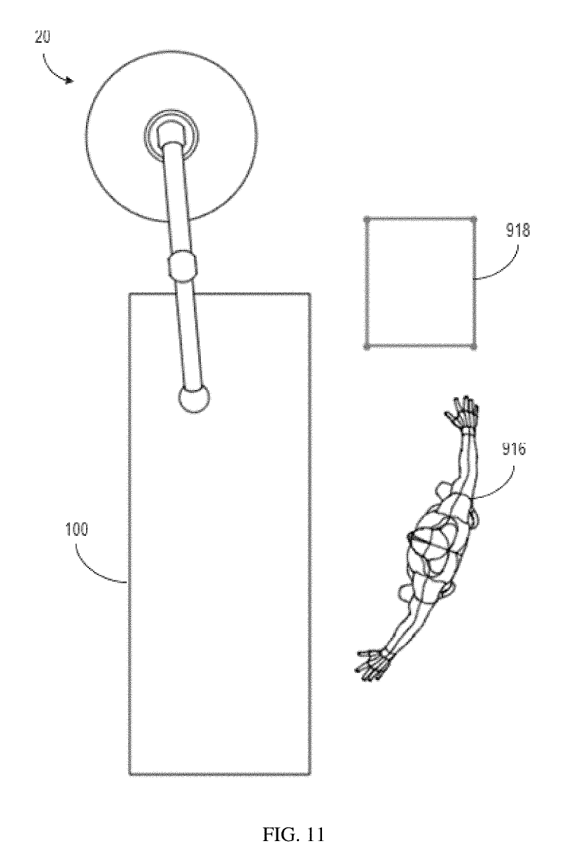

FIGS. 10A and 10B show the arm positioning device 20 relative to the gurney 100, in accord with aspects of the present disclosure. Although shown as being substantially centered relative to the gurney 100, the position of the arm positioning device 20 may vary depending on the arrangement of, for example, the user. For example, referring to FIG. 11, FIG. 11 shows the arm positioning device 20 relative to the gurney 100 and the user 916, in accord with aspects of the present disclosure. In addition, FIG. 11 shows the position of the arm positioning device 20 relative to the ultrasound machine 918. The arm positioning device 20 can be positioned to avoid interfering with the use of the ultrasound machine 918 while still providing an adequate range of motion to reach the patient on the gurney 100. Further, the arm positioning device 20 can instead be attached to the machine 918 and can provide information to the machine 918 during an examination, or as described previously, be attached to the ceiling via scaffolding or alternative in room stabilizing hardware.

Although described above as having a semi-passive configuration, alternatively, the arm pressure application assist device 20 may have an active or powered configuration. For example, the joints 914a-914c may include motors, servos, actuators, or the like that are controlled by the pedal 122 or other control devices, e.g. joystick(s). Accordingly, rather than dampening and locking the joints 914a-914c using the pedal 122, a sonographer can instead actively control the positioning of the arm 904 using a suitable control, such as the pedal 122 or other control devices rather than manually with the sonographer's hands. Additionally, rather than the transducer attachment 906 providing the pressure assistance, as discussed above with respect to the arch pressure application assist device 10, the powered movement of the entire arm 904 may generate the pressure assistance or may supplement the pressure assistance provided by the transducer attachment 906. In an active configuration, the pedal 122 (or control device) can be remote from the arm pressure application assist device 20 such that the sonographer can control the arm pressure application assist device 20 from a location remote to the subject.

### C. Passive Arm Positioning device

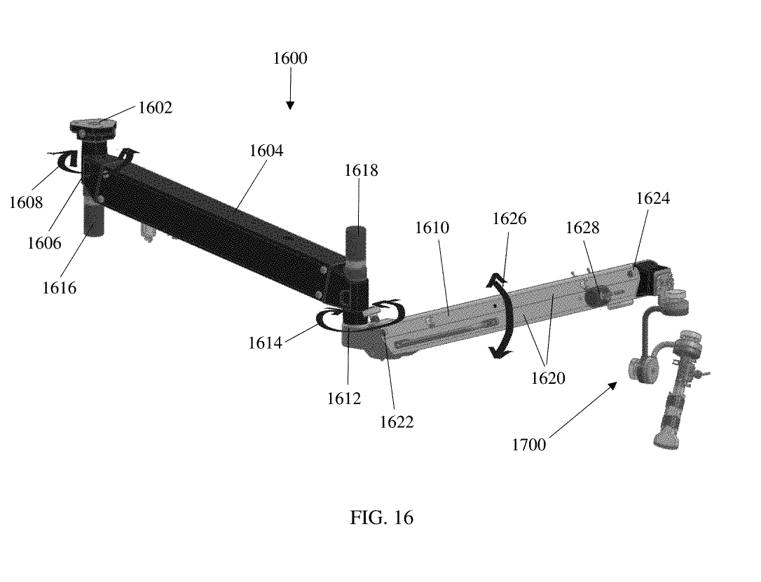

In an alternative embodiment, the arm positioning device and instrument assembly have a passive configuration. The arm positioning device and the instrument are manually positioned and any pressure is manually applied by the user. The arm positioning device may comprise motion assistance such that it cooperatively carries the weight of the arm where the user wants it moved so as to make the motion of the instrument as fluid and natural as possible. In some embodiments, the arm is at least partially counter-balanced, such as by springs and/or weight balancing, against gravity and lateral movement, such that it can be moved with minimum force by the user when the arm is in an unlocked state. Thus the user/operator does not have to compensate for gravity while moving the arm. Additionally, the counter-balancing may be configured to facilitate the arm positioning device to remain stationary when not moved or held by the user. FIG. 16 illustrates an exemplary passive arm positioning device 1600. With reference to FIG. 16, device 1600 comprises a foundation 1602 that provides an attachment to a support surface suitable to support the device 1600 during use. Suitable support surfaces include, but are not limited to, a ceiling, floor, wall, desk surface, cabinet surface, cupboard surface, a stand, or a medical instrument machine, such as an ultrasound machine. The stand may be a mobile or substantially stationary stand. Arm segment 1604 is attached to the support through joint 1606. Joint 1606 allows arm segment 1604 to move as indicated by arrow 1608. Arm segment 1610 is connected to arm segment 1604 through joint 1612. Joint 1612 allows arm segment 1610 to move relative to arm segment 1604 as indicated by arrow 1614. Locking mechanisms 1616 and 1618 increase the resistance to movement of joints 1606 and 1612, respectively, and may substantially prevent movement about the joint(s).

Arm segment 1610 comprises a plurality of bars 1620 and joints 1622 and 1624. The plurality of bars 1620 comprises at least 2 parallel bars and may comprise 4 bars. Joint 1622 allows arm segment 1610 to move in a plane perpendicular to the floor, as indicated by arrow 1626. The combination of the plurality of bars 1620 and joints 1622 and 1624 are configured to position the distal end of arm segment 1610 substantially perpendicular to the floor as the arm segment moves up and down. This allows the instrument assembly 1700 to maintain substantially the same orientation relative to the floor as the arm segment 1610 is raised and lowered. FIG. 16 illustrates an exemplary embodiment comprising two arm segments 1604 and 1610. However, a person of ordinary skill in the art will appreciate that the arm positioning device 1600 may comprise additional arm segments attached by additional joints, and that each joint may have an additional locking mechanism.

FIG. 16 also shows locking mechanism 1628 located on arm segment 1610. Locking mechanism 1628 increases the resistance to, and/or substantially prevents, movement of arm segment 1610 about joints 1622 and 1624. Locking mechanism 1628 may be grouped with locking mechanisms 1616 and 1618, such as in embodiments where locking mechanisms 1616, 1618 and 1628 may be activated or released independently of other locking mechanisms.

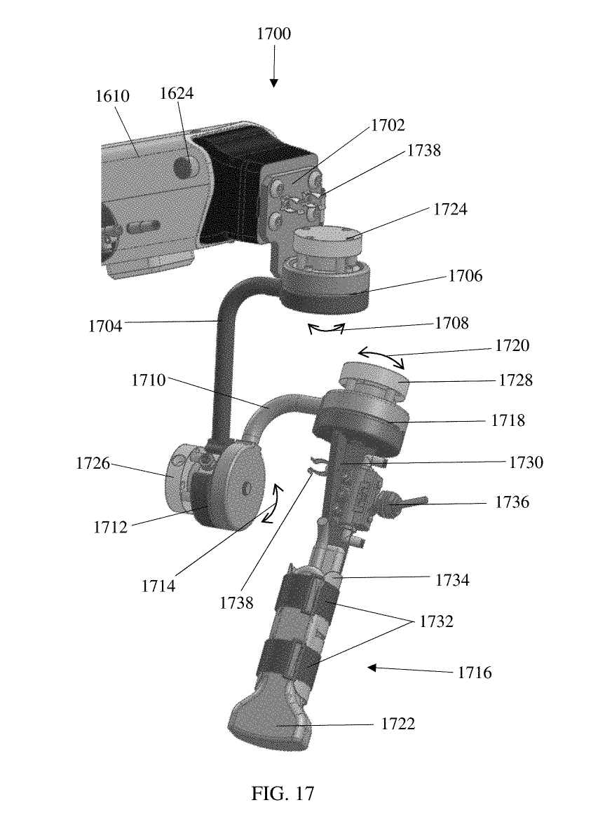

FIG. 17 provides an exemplary embodiments of the instrument assembly. With reference to FIG. 17, instrument assembly 1700 is attached to the distal end of arm segment 1610 through segment 1702. Segment 1702 is connected to assembly arm segment 1704 through assembly joint 1706, which allows assembly arm segment 1704 to move relative to segment 1702 as indicated by arrow 1708. Assembly arm segment 1704 is connected to assembly arm segment 1710 by assembly joint 1712. Assembly arm segment 1710 moves relative to assembly arm segment 1704 as indicated by arrow 1714. Instrument attachment assembly 1716 is attached to assembly arm 1710 through assembly joint 1718, which allows the instrument attachment assembly 1716 to move relative to assembly arm 1710 as indicated by arrow 1720. Assembly joints 1706, 1712 and 1718 allow fine control of an instrument 1722, such as a transducer, enabling accurate placement of the instrument on a patient. Instrument assembly 1700 also comprises locking mechanisms 1724, 1726, and 1728 that increase the resistance to movement of joints 1706, 1712, and 1718, respectively, and may substantially prevent movement about the joints.

Instrument attachment assembly 1716 comprises an arm segment 1730 onto which the instrument 1722, such as a transducer, is attached. In the exemplary attachment assembly illustrated in FIG. 17, instrument 1722 is a transducer attached to arm segment 1730 by straps 1732. The straps are any suitable strap that can hold the transducer to arm segment 1730 sufficiently tightly such that the transducer does not move when pressure is applied by the user and maintained by the locked arm. The straps may be elastic; may comprise a fastening mechanism such as a buckle; may comprise pliable bands and ratcheting straps; may comprise a hook and eye attachment, such as in a Velcro® strap; or a combination thereof. The instrument attachment assembly 1716 may optionally comprise a friction material 1734 that can be any material suitable to increase the friction between the transducer 1722 and the arm segment 1730 to further help prevent movement of the transducer relative to the arm segment. Other alternative instrument attachment assemblies, include, but are not limited to, a threaded clamp, a snap molding, a friction material, or a combination thereof.

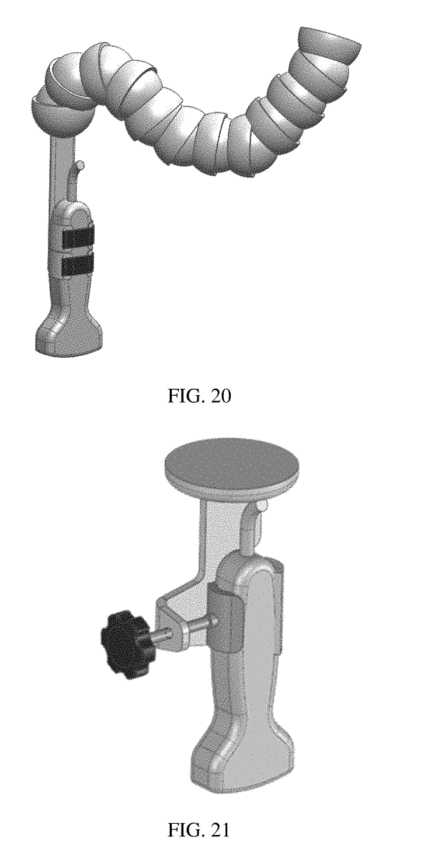

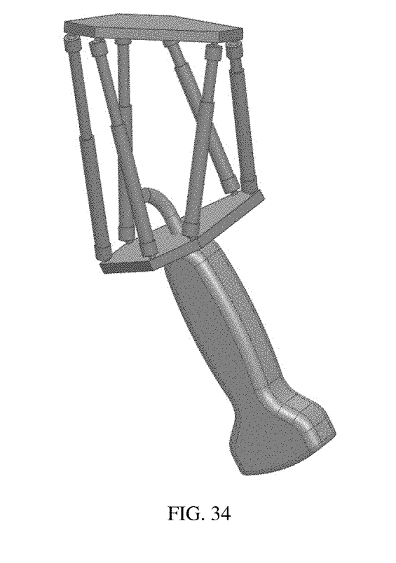

Alternative embodiments of an instrument assembly include, but are not limited to, an assembly comprising ball and socket joints, or a hexapod assembly (FIGS. 20 and 34, respectively). A hexapod assembly is a parallel mechanism comprising two rigid plates, connected with six links, although other numbers of links can be used. The six links between the two plates can each change their lengths by extending or compressing. A hexapod can provide six axes of motion in a compact mechanism. It is more rigid than a comparable serial mechanism, because the small devations at each joint are not additive; the overall deviation in the mechanism is less than the sum of the six individual deviations. Ball and socket joints offer some of the same advantages of parallel mechanisms (FIG. 20). They also can provide three axes of motion (yaw, pitch, and roll) in a simple, compact mechanism

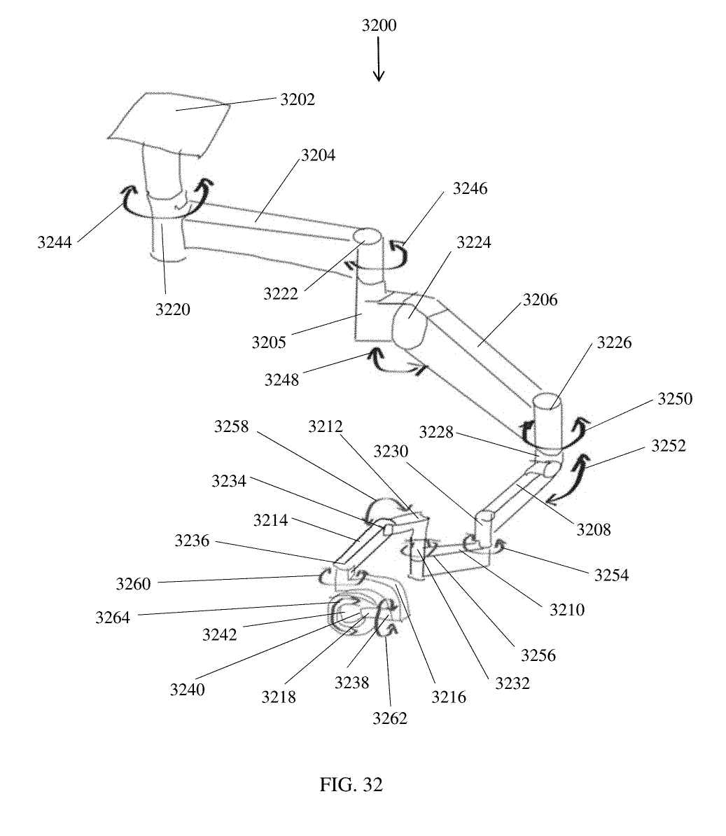

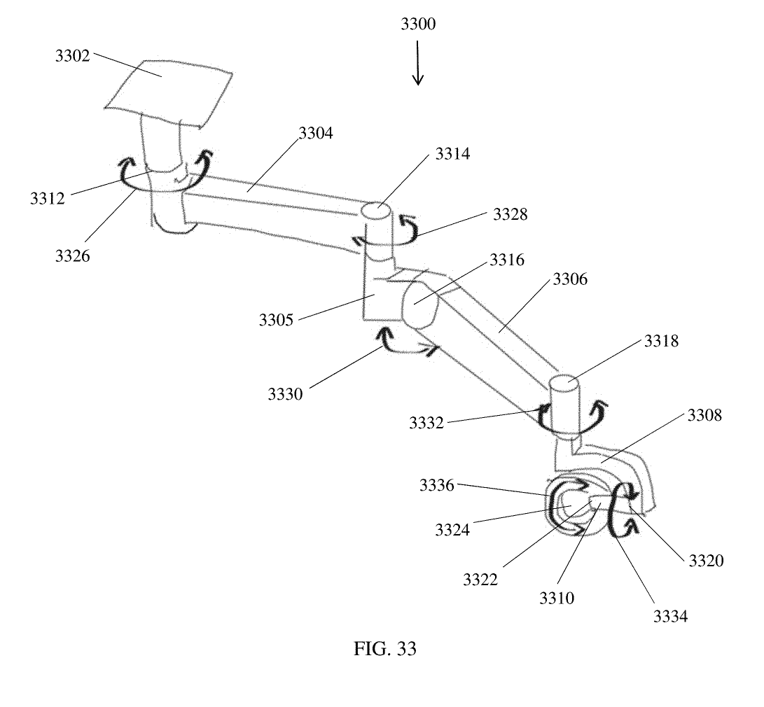

A person of ordinary skill in the art will appreciate that device 1600 and assembly 1700 comprise additional arm segments attached by additional joints, and that each joint may have an additional locking mechanism. FIGS. 32 and 22 provide exemplary embodiments of the positioning device having alternative numbers of arm segments. With reference to FIG. 32, positioning device 3200 comprises a ceiling mount 3202, arm segments 3204-3218, and joints 3220-3240. Instrument 3242, therefore, has 11 distinct degrees of freedom, illustrated by arrows 3244-3264. With reference to FIG. 33, positioning device 3300 comprises a ceiling mount 3302, arm segments 3304-3310, and joints 3312-3322. Instrument 3324, therefore, has 6 distinct degrees of freedom, illustrated by arrows 3326-3336.

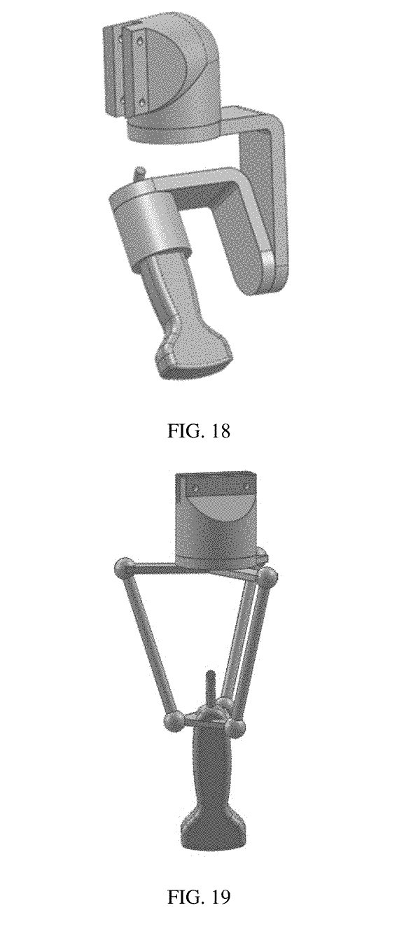

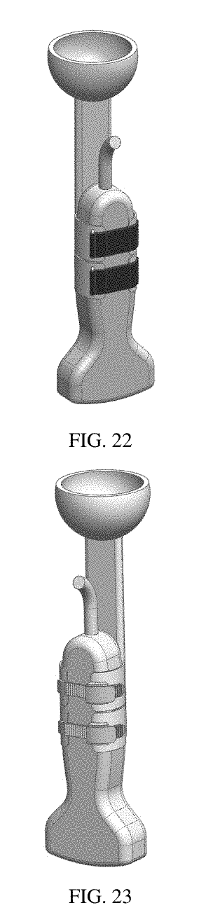

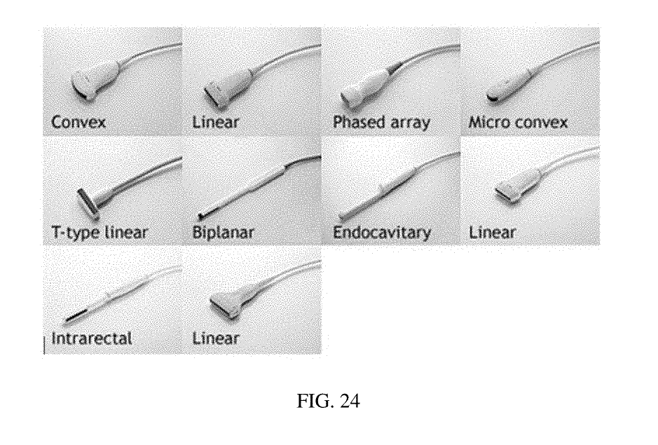

FIGS. 18-23 illustrate alternative embodiments of the instrument attachment assembly. FIG. 24 provides exemplary ultrasound probes, illustrating the variety of shapes and sizes of different probes.

Referring again to FIG. 17, instrument assembly 1700 may also comprise control 1736. In FIG. 17, control 1736 is located on arm segment 1730, but in other embodiments, control 1736 may be located on one of segments 1702, 1704 or 1710, or the control may be remote from the arm pressure assist device. In some embodiments, the control 1736 is located on the instrument attachment, such that it can be activated by the use's finger or thumb while they position the instrument and without them having to release the instrument and/or instrument attachment. Alternatively, control 1736 may be a pedal, switch or button that can be activated by the user's body part, such as a foot-activated pedal, button or switch, by the user's other hand, or by voice control. Control 1736 may activate one or more of locking mechanisms 1724, 1726 or 1728. In embodiments comprising a single control to lock the device's position, control 1736 may also activate locking mechanisms 1616, 1618, and 1628 shown in FIG. 16, and any other joints in the arm pressure assist device 1600. Control 1736 may be a switch, such as illustrated in FIG. 17. Alternatively, such as in embodiments where control 1736 is remote or separate from the arm pressure assist device, control 1736 may be any suitable actuator, such as a pedal, button, or an electronic control, and may be manually or computer controlled.

In some embodiments, such as a semi-active or active configuration of the instrument assembly 1700, where the instrument assembly 1700 extends the instrument 1722 towards a patient, such as to provide active compression assistance, the control 1736 can activate the compression assistance of the instrument assembly 1700, such as described above with respect to the pedal 122 and the fourth zone 124d. In such a configuration, the instrument assembly 1700 can include, for example, an actuator or similar device (e.g., a pneumatic or hydraulic actuator, an electric motor or servo, etc.) that extends the instrument 1722 towards to the patient to, for example, apply pressure to the patient with the instrument 1722 for compression during an ultrasound examination.

Instrument assembly 1700 may also comprise one or more clips 1738 that can hold an instrument cord (not shown).

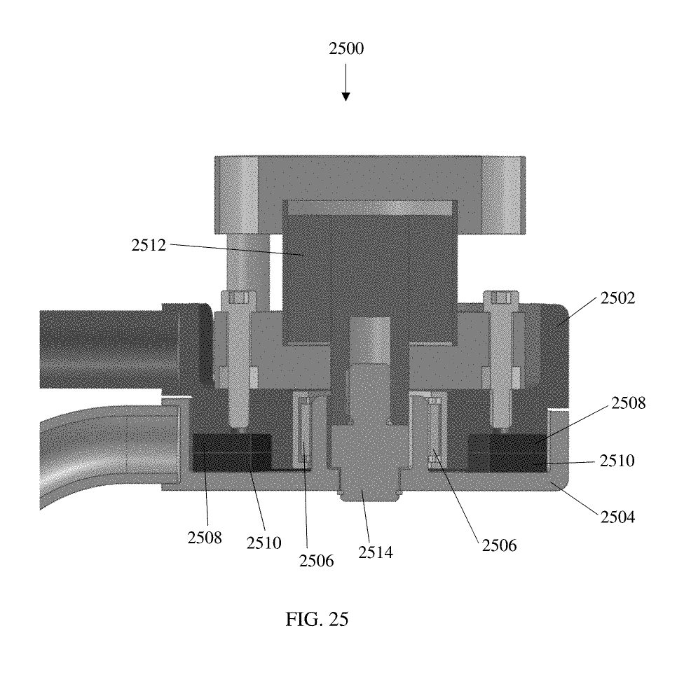

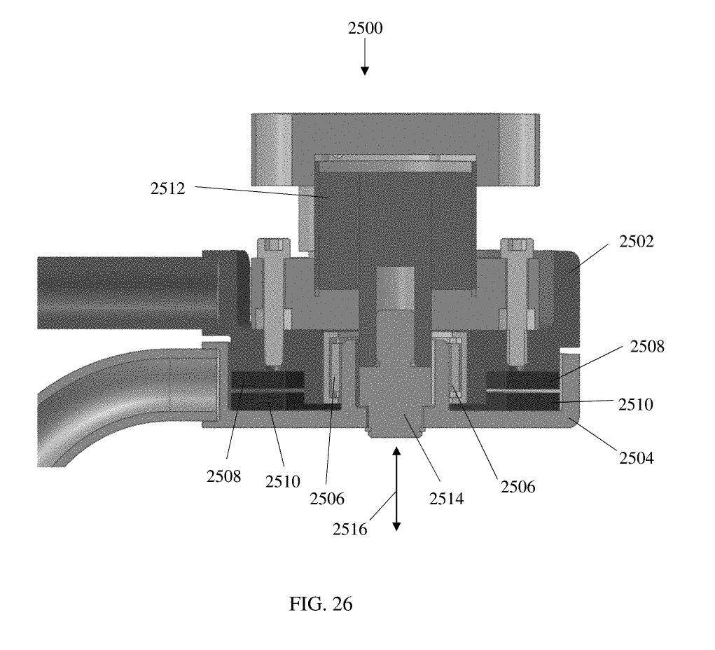

Referring again to FIGS. 16 and 17, locking mechanisms 1616, 1618, 1628, 1724, 1726, and 1728 may be any mechanism suitable to inhibit and/or substantially prevent movement at the respective joints and thereby inhibit and/or substantially prevent movement of the arm positioning device. Suitable locking mechanisms include, but are not limited to pneumatic, electrical, hydraulic, mechanical mechanisms, or a combination thereof. The locking mechanism may be a pneumatic or hydraulic friction grip brake similar to a disc brake pad or bicycle brake. The locking mechanisms may be located at each degree of freedom. In some embodiments, the device comprises a pneumatic locking mechanism. FIGS. 25 and 26 illustrate an exemplary pneumatic locking mechanism 2500 in a locked (FIG. 25) and unlocked (FIG. 26) configuration. With reference to FIGS. 25 and 26, the locking mechanism comprises an upper brake housing 2502 and a lower brake housing 2504. Bearings 2506 aid smooth movement of the upper brake housing 2502 relative to the lower brake housing 2504 when the locking mechanism is not engaged. Upper brake housing 2502 is connected to upper brake shoe 2508, and lower brake housing 2504 is connected to lower brake shoe 2510. When the locking mechanism is engaged, the upper and lower brake shoes 2508 and 2510 engage, thereby inhibiting and/or substantially preventing the relative movement of the upper and lower brake housing 2502 and 2504, and thereby inhibit and/or substantially prevent movement at the joint. Pneumatic locking mechanism 2500 also comprises pneumatic cylinder 2512. A shaft adapter 2514 is attached to the pneumatic cylinder 2512, and the lower brake housing 2504 is connected to the shaft adaptor at the distal end, such as by a retaining ring, such that movement of the pneumatic cylinder 2512 is translated into movement of the lower brake housing, as indicated by arrow 2516. When the locking mechanism 2500 is disengaged, pneumatic cylinder 2512 is pushed away from the upper brake housing 2502 by an internal spring (not shown), thereby placing the locking mechanism in an unlocked configuration. When the locking mechanism is to be engaged, the pneumatic cylinder 2512 pulls the lower brake housing 2504 toward the upper brake housing 2502, engaging the upper and lower brake shoes 2508 and 2510 and thereby inhibiting and/or substantially preventing movement of the upper and lower brake housings relative to each other.

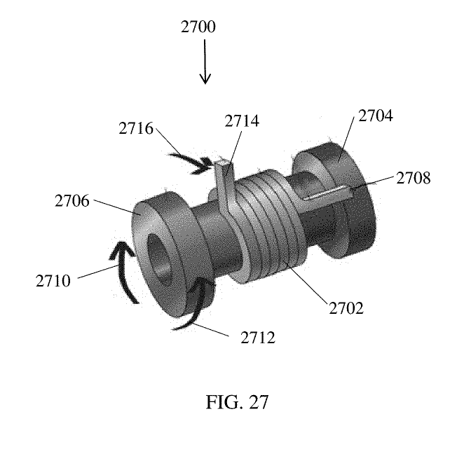

An alternative, or additional, locking mechanism comprises an electric actuator, such as a linear screw actuator. Electric actuators provide a force (either pushing or pulling) to squeeze the sandwiched parts, thus creating high frictional forces. Another alternative, or additional, locking mechanism is a wrap-spring mechanism 2700 (FIG. 27). With reference to FIG. 27, spring 2702 is loosely coiled around both hubs 2704 and 2706, and is attached to the first hub 2704 by tang 2708. When the second hub 2706 is rotated, it is free to move in direction 2710 because it is acting to unwind the spring 2702 and loosen its grip. However, if spring 2702 is rotated in direction 2712, it tightens the spring which grips both hubs 2704 and 2706, locking them together. The mechanism is dis-engaged by pushing on tang 2714 in the direction indicated by arrow 2716, which forces the spring into an unwound position, thereby allowing the hubs to freely rotate in both directions.

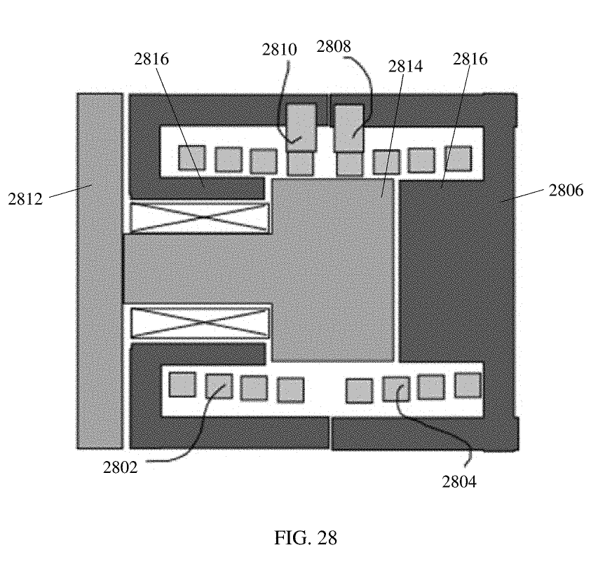

For a wrap-spring locking mechanism that can lock movement in both directions, a spring-based mechanism, such as a mechanism that include at least two springs is used (FIG. 28). With reference to FIG. 28, two springs 2802 and 2804 are attached to the same hub 2806 by tangs 2808 and 2810. Spring 2802 is a clockwise spring, and 2804 is a counterclockwise spring. In whichever direction hub 2812 is rotated, such action tightens one of springs 2802 and 2804, causing that spring to grip both shaft 2814 of hub 2812, and shaft 2816 of hub 2806. The hubs therefore are locked together. To release, or unlock, the locking mechanism, a control tang for each spring (not shown) would be pushed, thereby unwinding both springs and loosening their grips, allowing the hubs 2806 and 2812 to rotate freely.

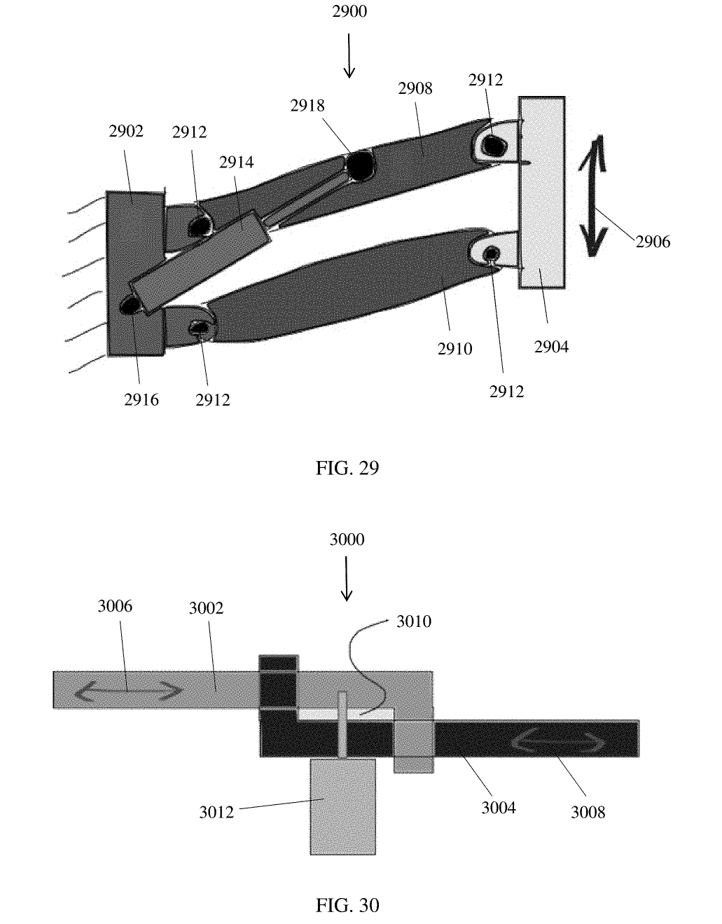

Another locking mechanism, often used in systems with four-bar linkages, is a fifth-link locking mechanism (FIG. 29). With respect to FIG. 29, four-bar linkage system 2900 comprises a first bar 2902 and a second bar 2904 that moves relative to the first bar 2902 in the direction indicated by arrow 2906. The movement of second bar 2904 is substantially parallel to first bar 2902. Bars 2908 and 2910 connect the first bar 2902 and the second bar 2904. Although the four-bar linkage system can be locked by locking at least one of the joints 2912 between the bars, typically a fifth-link locking mechanism is used. The fifth link 2914 is typically attached to the first bar 2902, by joint 2916, and one of the linking bars 2908 and 2910. In FIG. 29, the fifth link is attached to the upper bar 2908 though joint 2918. The fifth link is typically allowed to change its length, such as by telescoping, extending or compressing as the rest of the four-bar segment moves. To lock the segment, the fifth link is made rigid, preventing any length changes. In some embodiments, the fifth link is a locking gas spring. Typically, these devices can change their length, but they can be locked in any position by pressing a button on the side. This button is often actuated by a small solenoid. However, gas springs are usually designed to provide some resistance top motion, for example, to prevent a door from swinging shut quickly. While this may be advantageous in an embodiment where the four-bar linkage system is being used to counter-balance gravity, it can also add additional drag for a user trying to position the positioning device.

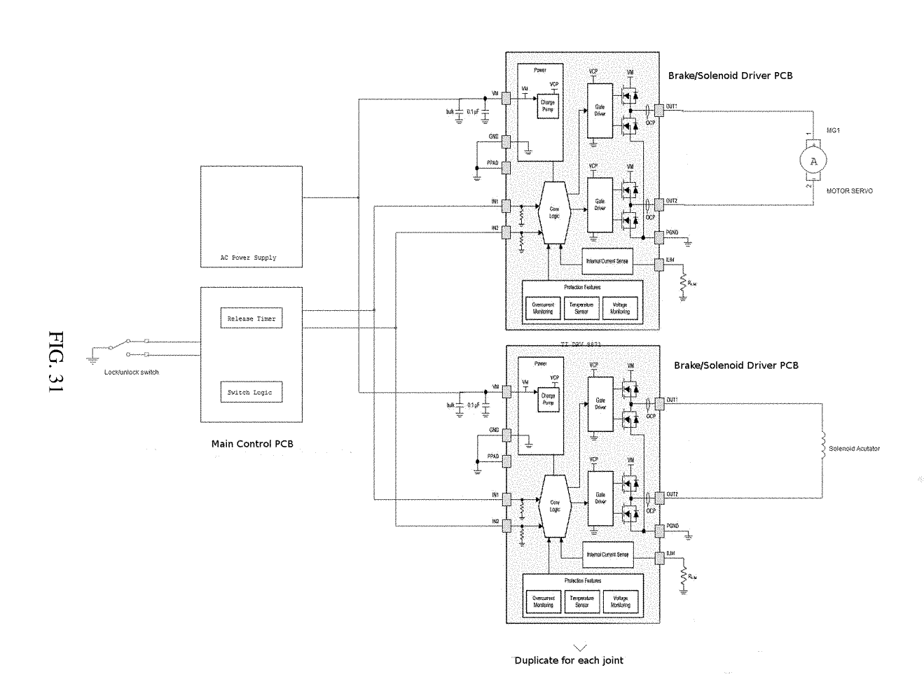

An alternative to the locking gas spring, is a sliding lock system 3000, shown in FIG. 30. With reference to FIG. 30, sliding lock 3000 comprises two freely sliding members 3002 and 3004, which can move in the directions shown by arrows 3006 and 3008, respectively. A friction material 3010 is located between the two members 3002 and 3004. When linear actuator 3012 is activated, member 3002 is pulled toward member 3004, sandwiching friction material 3010 between them, and thereby inhibiting and/or substantially preventing movement of the two members relative to each other. When used with the four-bar locking system 2900, the lock is prevented from extending or compressing, thereby preventing movement of bar 2908 relative to bar 2902, and thus locking the system 2900 in position.

Another alternative mechanism is a ball and socket joint assembly with an internal locking mechanism.

With reference again to FIGS. 16 and 17, once the instrument 1722 is in a desired position, locking mechanisms 1616, 1618, 1628, 1724, 1726 and 1728 are engaged to lock all the joints in position, thereby substantially preventing the instrument from moving and, if necessary, allowing the user to stop applying pressure. Any pressure that was initially applied by the user is substantially maintained by the locked joints in the positioning device.

In alternative embodiments, the user positions the instrument in the correct position without applying pressure and locks the arm segments and instrument assembly in that position. The user then activates a control, such as a pedal, to extend the instrument toward the patient, as is previously described embodiments. In this way, the positioning device both exerts and maintains the pressure, thereby further limiting the risk of traumatic injury to the user.

All the joints in the positioning device 1600 may be locked in position substantially at the same time upon activation of a single control, such as a single switch or pedal. Alternatively, joints may be locked individually, in groups, or in a combination thereof. In some embodiments, joints proximal to the foundation, such as joints 1606, 1612, 1622, and 1624, are locked as a first group, and the joints in the instrument assembly 1700, such as joints 1706, 1712 and 1718, are locked as a second group, separate from the first. Individual joints and/or groups of joints may be locked by separate controls, such as separate switches and/or pedals, or they may be sequentially locked by one control. For example, a single switch or pedal is moved to a first position to lock a first joint or group of joints, and then moved to a second position to additionally lock a second joint or group of joints (for example, see FIG. 8). Such a configuration allows a user to locate the instrument, such as a transducer, in approximately the correct position and lock the arm segments 1604 and 1610 in position. The position of the instrument can then be finely adjusted before the instrument assembly is locked in position. As previously described with reference to other embodiments of the positioning device, in an alternative embodiment the default state of the arm and/or instrument attachment may be the locked position, and the control, such as a switch or pedal, may be activated to release the locking mechanism(s) to allow movement.

In some embodiments, the locking mechanisms are electrically controlled. The electronic control will receive input from the user indicating whether the system should be locked or unlocked, and generate electrical outputs to the locking mechanisms, such as actuators, based on the inputs. Additionally, the electrical system may also comprise a timing mechanism to release the locking mechanisms after they have been engaged for a predetermined length of time, to release a patient from the instrument. For example, for sonography applications, this will prevent the patient from being exposed to excessive amounts of ultrasound energy.

In some embodiments, a main control circuit board may be part of the mount assembly. It receives input from the user controls, such as pedal or switch, and sends output signals to the brake driver boards. It also contains the safety release timer. The safety timer starts every time the user sonographer locks the joints. If the joints remain locked for an excessive length of time (as determined in the product specification for each application) logic circuitry reverses the current to the locking mechanisms, causing the brakes to disengage.

The brake/solenoid circuit board may be installed immediately adjacent to each of the locking mechanisms, i.e. there will be one of these boards at each of the mechanical joints (FIG. 31). Typically, the locking mechanism is driven forward until it stalls. The locking mechanism is selected to match the mechanical system, in a way that ensures that the locking mechanism stalls just at the point where it has developed the correct level of braking force. Unlocking will occur in a similar way, driving the locking mechanism backwards until it stalls. However, locking mechanisms comprising a linear drive screw are not back-drivable, so they will hold the braking force even when there is no power applied to the locking mechanism. In other words, the locking mechanism only moves the brake into position; once it's in place, the mechanical system will bear the entire load and the power to the locking mechanism is turned off. The brake/solenoid circuit board can also be used to drive solenoids for locking four-bar linkages. Electrically, the task is very similar, supply DC power to a device until a predetermined current threshold is reached.

The user input system can be any user input system suitable for receiving input from a user and setting the locked and unlocked states of the locking mechanisms. The user input system may be a switch, such as a two way switch; a pedal, such as a foot pedal; or a computer input device, such as a keyboard or mouse.

### D. Force Amplifying Device

In any embodiments of the positioning device, the instrument assembly may comprise a force amplifying apparatus. When a user applies a force, typically a mild or gentle force, to a force amplifying apparatus, the apparatus applies a greater force to the subject. For example, in embodiments concerning sonography, a force amplifying apparatus reduces the amount of downward force that the sonographer needs to apply to obtain an ultrasound image and/or perform procedures comprising ultrasound. The force amplifying apparatus may comprise a force detector that detects the amount of force applied by the user. The force detector is in communication with a force applicator, which applies the desired force to the subject. The communication may be through a computer; a mechanical communication, such as through gearing and/or leverage; an electronic communication; or a combination thereof. And amplification may be achieved by computer programming, and/or by mechanical and/or electronic gearing and leverage ratios.

The force may be applied by any suitable technique, such as electronic actuator, a pneumatic actuator, a hydraulic actuator, a pneumatic or hydraulic piston, or a combination thereof. In embodiments, comprising a pneumatic or hydraulic system, the force detector may be in communication, either electronically or mechanically, with a pneumatic or hydraulic pump, so as to activate the pneumatic or hydraulic actuator or piston.

### E. Applications of the Positioning device

The positioning devices of the present disclosure can be used during various ultrasound examinations, such as, but not limited to, ultrasound applications of the abdomen, heart, breast, chest, and/or vascular system. However, although described primarily with respect to an ultrasound examination, the positioning devices of the present disclosure can be used for various additional purposes related to ultrasound examinations and/or other examinations or procedures. Accordingly, although described primarily with respect to being controlled by a sonographer, the positioning devices can be controlled by various users, such as various medical professionals, technicians, operators, clinicians.

With respect to the additional uses, the positioning devices can be used in the delivery of therapeutic agents and manipulation of the patient, during or independent of performing an ultrasound examination. As specific examples, the positioning devices can be used in the manipulation of a patient to move gas bubbles within a patient, agitate a fetus, and assess Murphy's sign, to list a few examples. With respect to the delivery of therapeutic agents, advances have been made that allow the administering of agents into a patient's system, and even targeting the agents to a specific location within the patient. Such agents may be micro bubbles that have a specific therapeutic agent contained within. Ultrasound energy can be administered to a precise point of the patient, which causes the micro bubbles that pass through the point to burst, releasing the therapeutic agent at the precise location. In such procedures, control over the pressure of the transducer and its position on the patient is critical to provide the ultrasound energy at the precise point for delivery of the agents. The positioning devices assist a user in managing the delivery of the therapeutic agents to the specified region(s) of the patient. In addition, there can be a combined effect that allows for ultrasound imaging to ensure the correct position of the transducer, followed by application of the ultrasound energy to burst the micro bubbles. According to the potential inability of sonographers to manually control the pressure at a specific level for an extended period of time with their arms, the positioning devices of the present disclosure now allow for procedures that were previously not possible, or allow for an increased performance quality during certain procedures.

The positioning devices can further aid during other procedures and/or examinations. For example, the positioning devices can act to provide traction and act as a supporting device during surgery or any medical procedure/intervention. In such cases, the positioning devices can provide a pressure and/or a traction role during surgery. The transducer attachments 112 and 906 can be replaced with other components, or be modified to allow for the addition of other components, to provide both pressure and traction. The ability to control the angle of the positioning device allows for a more versatile positioning of pressure and traction. Further, the precise and dynamic control of the pressure and/or traction during a procedure, as compared to, for example, a static support device or a user, allows for specific control that otherwise would not be possible. It also allows a surgeon or other interventionist to obtain the functional use of their feet during a procedure to control the arm and enable an additional opportunity for control or intervention within a procedural field improving procedural performance.

As an alternative to a transducer, the positioning devices can instead have a blunt instrument connected to the transducer attachments 112 and 906 that allows the positioning devices to provide rhythmic pressure to assist in cardiac events. Although current devices exist that provide rhythmic pressure for cardiac events, such devices are static and do not allow for the angled positioning of the blunt object used to provide the pressure. According to the positioning devices of the present disclosure, the devices can be controlled to provide rhythmic pressure at specific locations on a patient and according to specific angles and degrees of pressure. The additional benefit of the present device would allow for manual control and guidance of pressure during cardiac events allowing a rescuer to administer intentional and guided pressure without the use of his hands enabling greater efficiency and preventing exhaustion.

The positioning devices of the present disclosure, and particularly the arm positioning device, can also be used to administer agents to a patient during examinations and/or procedures that would otherwise prohibit such administering. For example, in embodiments where a needle or probe is introduced to a patient, the needle or probe is received by a suitable instrument attachment and can linearly advanced toward the patient by the instrument assembly and/or maintained in a position by locking the positioning device once the needle or probe has been placed by the user. For example, the arm positioning device 20 can be operated to administer an agent to a patient during an examination, besides an ultrasound examination, such as during a magnetic resonance imaging (MRI) examination or a computerized tomography (CT) scan. Such examinations typically would not permit the administering of an agent to a patient based on the conditions associated with the examinations, i.e. due to, but not limited to, high radiation or magnetic field environments potentially harmful to a healthcare professional. However, the positioning devices of the present disclosure can be remotely operated by the control devices, such as the pedal 122, by an operator that is remote from the patient. This can allow a practitioner (i.e. interventionist) to apply a controlled pressure, administration or insertion of a needle at a dedicated and specific angle to a highly specific depth within a patient. This is particularly useful for minimally invasive procedures during which a predesignated course is determined by an interventionist to obtain access into a subject, such as into a tumor, mass or abscess. During imaging the practitioner can observe from outside the room the location of the needle tip advancing it deliberately without concern for changing direction or angle of the needle due to the locking of the joints and the strict linear pressure of the pressure assistance.

Moreover, the ability to apply a rotational movement as the transducer 114 is extended towards the patient allows the positioning device to assist in additional procedures. By way of example, and without limitation, the positioning devices can assist in routing catheters through patients, with the rotational movement allowing the catheter to pass through bends within the patient.

The embodiments of the apparatus and method disclosed herein will benefit sonographers, the medical centers, and patients in multiple ways. By eliminating the primary risk factor for repetitive stress injuries, sonographers will be able to work consecutive, longer, and more productive hours. Their careers will be longer and more productive and require less time off for medical care. Medical centers will benefit from the apparatuses and methods disclosed herein. The positioning device will increase the sonographers' efficiency and accuracy, thereby decreasing the number of sonographers required in a department to perform the required examinations, as well as increasing the daily quantity of examinations performed. Furthermore, the medical centers benefit from the elimination of time taken off for injuries, early retirement, medical expenses, and recruiting and hiring new/temporary employees. Patients will benefit from the apparatuses and methods disclosed herein by receiving examinations from sonographers that are not fatigued or uncomfortable, compromising the efficacy of the examination. Furthermore, sonographers that have longer and more productive careers have improved expertise and can perform more efficient and accurate examinations.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the invention. It is also contemplated that additional embodiments according to aspects of the present invention may combine any number of features from any of the embodiments described herein.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A device, comprising a plurality of locking mechanisms configured to inhibit and/or substantially prevent movement of an instrument assembly relative to a patient, and at least one controller connected to the plurality of locking mechanisms, configured to lock and unlock the locking mechanisms.

2-40. (canceled)
